(19) United States
(12) Patent Application Publication
(10) Pub. No.: US 2007/0265650 A1
(43) Pub. Date: Nov. 15, 2007

(54) Device for separating the epithelial layer from the surface of the cornea of an eye

(76) Inventors: Ioannis Pallikaris, Gazi of Heraklion (GR); Harilaos Ginis, Heraklion (GR); Scott M. Hampton, Cumming, GA (US); Stephen Woods, Hitchin (GB)

Correspondence Address: BRINKS HOFER GILSON & LIONE, P.O. BOX 10395, CHICAGO, IL, 60610, US

(21) Appl. No.: 11/699,919

(22) Filed: Jan. 30, 2007

Related U.S. Application Data
(63) Continuation-in-part of application No. PCT/US05/28188, filed on Aug. 8, 2005.
(63) Continuation-in-part of application No. 10/971,727, filed on Oct. 22, 2004.
Application No. 10/971,727 is a continuation-in-part of application No. 10/098,167, filed on Mar. 12, 2002, now Pat. No. 7,004,953.
Application No. 10/098,167 is a continuation-in-part of application No. 09/911,356, filed on Jul. 23, 2001, now Pat. No. 7,156,859.
(60) Provisional application No. 60/599,368, filed on Aug. 6, 2004.

(51) Int. Cl. A61F 9/007

(52) U.S. Cl. 606/166

(57) ABSTRACT

A separator is disclosed for moving a separated portion of an eye. The separator includes an upper surface. A lower planar surface is oriented at an angle with respect to the upper surface. The upper surface and the lower planar surface define an edge therebetween having a structure to remove a portion of an eye. The upper surface includes a structure to receive the removed portion of the eye and to roll the removed portion.

### REFERENCE TO EARLIER FILED APPLICATIONS

Applicants claim the benefit as a continuation-in-part under 35 U.S.C. § 120 of PCT Patent Application No. PCT/US2005/028188, filed Aug. 8, 2005 (Pending), which claims, under 35 U.S.C. § 119(e), the benefit as a continuation-in-part of priority of the filing date of Aug. 6, 2004, of U.S. Provisional Patent Application Ser. No. 60/599,368, filed on the aforementioned date, the entire contents of which are incorporated herein by reference, and in addition the present application is a continuation-in-part application of U.S. patent application Ser. No. 10/971,727, filed on Oct. 22, 2004, which is a continuation-in-part application of U.S. patent application Ser. No. 10/098,167, filed on Mar. 12, 2002, which is a continuation-in-part application of U.S. patent application Ser. No. 09/911,356, filed Jul. 23, 2001, the entire contents of each of which are incorporated by reference herein.

### BACKGROUND

Surgical procedures, such as LASIK (Laser-Assisted In Situ Keratomileusis), LASEK (Laser Epithelial Keratomileusis), PRK (Photo-Refractive Keratectomy) and EPI-LASIK, are intended to reduce a person's dependency on glasses or contact lenses. To correct the person's vision, a laser is typically used to permanently change the shape of a stroma layer of the eye. An outer layer of the eye, the epithelial layer, may be moved or removed so that the laser can reach the stoma. A device, such as an epi-keratome or microkeratome, may be used to move the epithelial layer to reveal the stroma.

When the epithelial layer is moved, a flap may be created that is folded back to reveal the stroma. The revealed stroma may expose nerves in the eye to the environment. Due to the exposed nerves, the person may experience pain during the healing period after the procedure. To aid in the healing process, the removed epithelial layer may be replaced over the exposed stroma to cover the nerves after the laser process is completed. The replaced epithelial layer then typically disintegrates as a new epithelial layer grows back. A problem may occur in that it may be difficult for a surgeon to handle the folded epithelial layer, so that it may not be easy or possible to replace the epithelial layer after the surgery. Also, the epithelial layer may be damaged as it is folded or unfolded.

### BRIEF SUMMARY

A method is used for processing an eye of a patient for a corrective procedure. A separator having an edge is provided. An epithelial layer of the eye of the patient is penetrated with the edge. The separator moves relative the eye of the patient. At least a portion of the epithelial layer is separated as the separator moves. At least a portion of the separated epithelial layer is rolled during the separating.

A separator is disclosed for moving a separated portion of an eye. The separator includes an upper surface having a curved shape. A lower planar surface is oriented at an angle with respect to the upper surface. The upper surface and the lower planar surface define an edge therebetween having a structure to remove a portion of an eye.

The separation process with respect to the present invention may provide several advantages. For example, the separator may separate the epithelium without substantial epithelial cell loss to the separated portion. Yet other advantages of the present invention may include the simplicity of the procedure, the speed of the procedure and the fact that it may require less surgeon training than other techniques. Another advantage is that a rolled epithelial layer may help prevent rupturing of the epithelial layer while waiting to be replaced on the corneal substrate. The rolled epithelial layer may also be advantageous because a surgeon need not further handle the removed epithelial layer before ablating the eye.

### BRIEF DESCRIPTION OF THE DRAWINGS

FIG. 1A is a diagram showing a side view of an eye and an epithelial separator device with a separator located in a first position according to the preferred embodiments.

FIG. 1B is a side view of an embodiment of the separator used with the epithelial separator device of FIG. 1A.

FIG. 1C is a top, perspective schematic view of an embodiment of separator support to be used with the epithelial separator device of FIG. 1A.

FIG. 2 is a diagram showing a top view of the eye and the separator located in a first position according to the preferred embodiments.

FIG. 3 is a diagram showing a side view of the eye and the separator located in a second position according to the preferred embodiments.

FIG. 4 is a diagram showing a top view of the eye and the separator located in a second position according to the preferred embodiments.

FIG. 5 is a diagram showing a side view of the eye and the separator located in a third position according to the preferred embodiments.

FIG. 6 is a diagram showing a top view of the eye and the separator located in a third position according to the preferred embodiments.

FIG. 7 is a diagram showing a side view of the eye and the separator located in a fourth position according to the preferred embodiments.

FIG. 8 is a diagram showing a top view of the eye and the separator located in a fourth position according to the preferred embodiments.

FIG. 9 is a diagram showing a top view of the eye and the separator located in a fifth position according to the preferred embodiments, the separator is retracted after epithelial separation and the applanator and edge are formed at a higher position than the applanator and edge of FIGS. 1-8 so that a smaller disk is separated.

FIG. 10 is a diagram showing a top view of the eye with the separator removed.

FIG. 11 is a diagram showing a top view of the eye after ablations is performed with a laser.

FIG. 12 is a diagram showing a top view of the eye with the epithelium replaced on the eye.

FIG. 13 is a diagram showing a top view of the eye with the epithelium smoothly stretched into place.

FIG. 14 is a diagram showing a side view of the eye and the epithelial separator device including a rotating drum.

FIG. 15 is a diagram showing a front view of the eye and the epithelial separator device including the rotating drum.

FIG. 16 is a diagram showing a top view of the eye and the epithelial separator device including the rotating drum.

FIG. 17 is a diagram showing a drum according to one embodiment.

FIG. 18 is a diagram showing a drum according to another embodiment.

FIG. 19 is a diagram representing a side view of a separator removing the epithelial layer from the corneal surface of the eye.

FIG. 20 is a diagram showing a perspective view of a known blade.

FIG. 21 is a diagram showing a side view of a separator's leading edge according to an embodiment.

FIG. 22 is a diagram showing a side view of a separator's leading edge according to another embodiment.

FIG. 23 is a diagram showing a side view of a separator's leading edge according to yet another embodiment.

FIG. 24A shows a top perspective view of a second embodiment of an epithelial separator device according to the present invention.

FIG. 24B shows a bottom perspective view of the epithelial separator device of FIG. 24A.

FIG. 25 is a side view of an embodiment of the separator used with the epithelial separator device of FIGS. 24A-B.

FIG. 26 shows a perspective view of an embodiment of a guard to be used with the epithelial separators devices of FIGS. 1A-B and 24A-B.

FIG. 27 shows a top perspective view of the epithelial separator device of FIG. 24 when employing an embodiment of an applanator at a first position.

FIG. 28 shows a top perspective view of the epithelial separator device of FIG. 24 when employing the applanator of FIG. 27 at a second position.

FIG. 29 shows a bottom perspective view of the epithelial separator device of FIG. 24 when employing the applanator of FIG. 27.

FIG. 30 is a diagram showing a side view of a separator according to another embodiment.

FIG. 31A is a diagram showing a side view of a separator according to yet another embodiment.

FIG. 31B is an enlarged side view of a portion of a leading edge of the separator shown in FIG. 31A.

FIG. 31C is an enlarged side view of a portion of a leading edge of another embodiment of a separator.

FIG. 31D is an enlarged side view of a portion of a leading edge of another embodiment of a separator.

FIG. 31E schematically shows the separation process using the separator of FIG. 31D, wherein enlarged views of beginning and final stages of the separation process are shown as well.

FIG. 32A is a side view of a separator according to yet another embodiment.

FIG. 32B is the side view portion of the separator shown in FIG. 32A, with additional dimensions being shown.

FIG. 32C is a side view of a separator according to yet another embodiment.

FIG. 32D is a side view of a separator according to yet another embodiment.

FIG. 32E is a side view of a separator according to yet another embodiment.

FIG. 32F is a side view of a separator according to yet another embodiment.

FIG. 32G is a side view of a separator according to yet another embodiment.

FIG. 32H is a side view of any of the separators of FIGS. 32A-32G or any other separator that acts to roll the epithelial layer as the separator moves across the eye.

FIG. 32I is a top view of any of the separators of FIGS. 32A-32G or any other separator that acts to roll the epithelial layer as the separator moves across the eye.

FIG. 33 is a diagram showing a perspective view of another embodiment of a separator, which is in the form of a wire.

FIG. 34 shows a perspective view of a machine that is used to condition a separator according to one embodiment.

FIG. 35A shows a front view of the machine of FIG. 34 including the separator.

FIG. 35B schematically shows a side view of the machine of FIG. 34 including the separator.

FIG. 36 shows a side view of one embodiment of a separator device that has two rotating drums.

FIG. 37 shows a top view of the separator device of FIG. 36.

### DETAILED DESCRIPTION

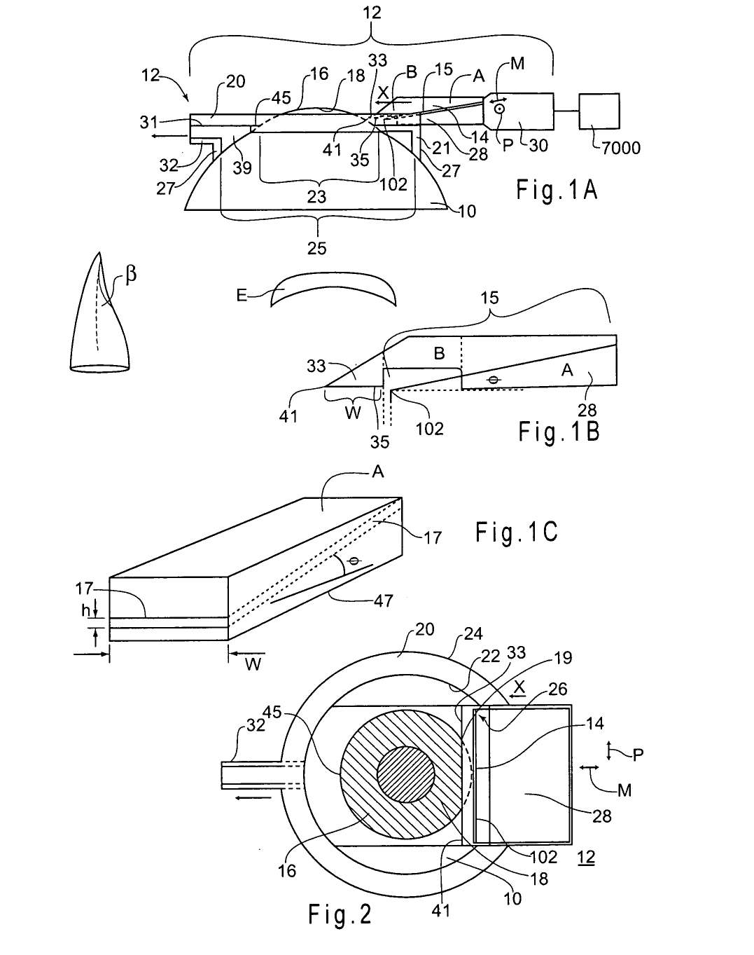

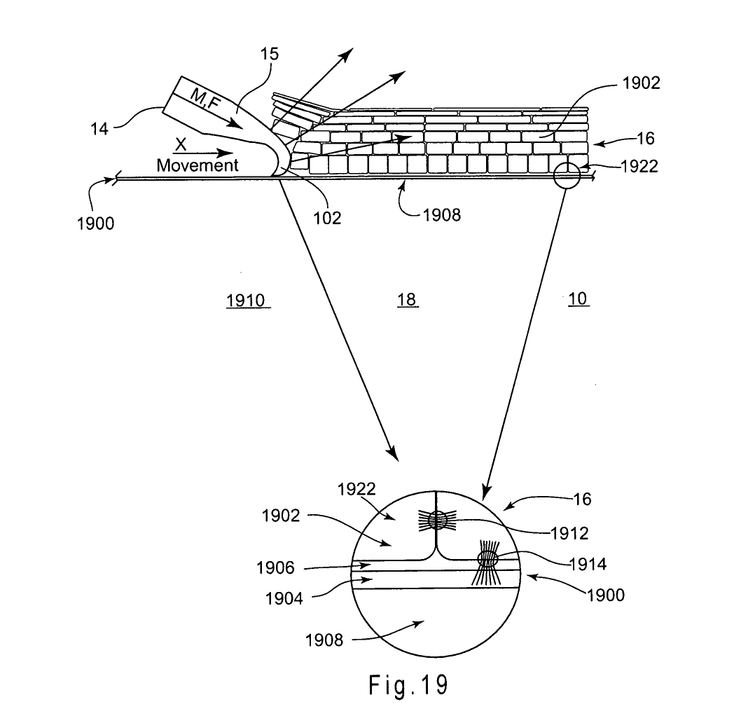

As part of process for improving an eye's 12 vision an epithelial separator device 12 (as shown in FIGS. 1-9) separates at least a portion of an epithelial layer or epithelial material 16 positioned on a corneal substrate 18. As defined throughout the present patent application, the corneal substrate 18 as shown in FIG. 19 includes at least the corneal stroma 1910 and can further include one or more of: 1) Bowman's layer 1908 and 2) Basal membrane 1900, which will be described in more detail later in the present patent application. Accordingly, the various separation processes described herein with respect to the present invention can be performed either when Bowman's layer and/or the Basal membrane are positioned upon the corneal stroma or when the Bowman's layer and/or the Basal membrane are absent from the eye in question. Under normal circumstances, separation occurs between 2 main layers of the Basal membrane. The latter situation typically occurs, for example, when the eye has previously been subjected to a surface ablation surgery, such as PRK, LASEK or the one described herein with respect to the present invention. Likewise, and at least partially for this reason (and also because separation may occur at different junctures, though not intending to be attached to one theory, as it is believed that separation often occurs between the two main layers of basal membrane as discussed below), when the present application refers to a separated epithelium or epithelial layer or material it is with the understanding that Bowman's layer and/or Basal membrane material may be attached thereto as well.

While typically used for preparing the eye for laser ablation such as correction, the separator device 12 can be also used for other surgical procedures, such as lens insertion. In ablation procedures, the portion of the epithelial layer 16 is removed because it overlies the area of the eye 10 requiring correction and the physician requires unobstructed access to this site.

The thickness of the separated portion of the epithelial layer 16 preferably corresponds to the full thickness of the layer 16. While it is also possible to adjust the separator device 12 so that the separated portion has a thinner thickness, this is not ideal for laser ablation procedures since a thin layer of the epithelial layer 16 would still remain on the cornea and would need to be removed for laser ablation to be performed. The separator device 12, once properly adjusted, is able to remove the entire epithelial layer 16 during a single crossing of the cornea as will be described later in this patent application.

The separation process described above and below with respect to the present invention provides several advantages over LASIK and LASEK. For example, in contrast to compared with prior techniques that employ alcohol, such as LASEK, the device 12 separates the epithelium without substantial epithelial cell loss to the separated portion. In the case of an epithelial layer 16 that is entirely removed from the corneal stroma, there is an epithelial cell loss that is often less than 5-10% which ensures that the layer 16 is viable. The low epithelial cell loss achieved by device 12 therefore achieves a more viable epithelial layer 16 after replacement on the corneal substrate 18, when. Such a more viable epithelial layer 16 may provide improved healing for the eye 10 when compared with LASEK after the laser ablation is performed and the epithelial layer 16 is placed back on the corneal stroma or Bowman's layer.

As compared to LASIK, the present invention does not remove any substantial amount of corneal stromal material (and ideally none) from the corneal substrate 18. The failure of the present invention to cut into and remove corneal stroma material, as with the LASEK sharp blade, results in the corneal stroma being more mechanically sound when compared with the stroma material remaining in a LASIK procedure.

Likewise, compared to the LASIK procedure, the failure to cut into and remove corneal stroma material also presents a smaller risk of creating optical irregularities. In contrast the procedure described herein, the microkeratome used in LASIK removes a flap including a substantial piece the corneal stroma, which is more “permanent” tissue, and replaced the flap on the eye 10 after ablation. If the microkeratome takes out a different amount of corneal stroma than required to accomplish the abalation/correction procedure, then the patient may have to live with this damage forever or undergo a further corrective procedure such as corneal transplantation. Thus, in sum, the cornea stroma, prior to laser ablation, is much more mechanically sound in structure and its optical properties are not significantly affected after the layer 16 is removed by invention's procedure when compared with the case when a LASIK flap is cut for the same eye.

Another advantage of the present invention is that the consequences of an error being made using the present invention are not as dire in other surgical techniques, such as LASIK. As mentioned previously, if the corneal stroma is incorrectly operated on during the LASIK procedure, permanent vision impairment may result or further surgery may be required. In contrast there is no serious problem if something goes wrong in the separation of the epithelial flap while using the present invention; rather, the surgeon can simply discard the flap and it will grow back again in a matter of days.

Yet other advantages of the present invention include the simplicity of the procedure, the speed of the procedure and the fact that it requires much less surgeon training than other techniques.

Some embodiments of the separator devices 12′, 2700, such as shown in FIGS. 14-18 and FIGS. 35-36, include yet even further advantages via the addition of a holder, such as a drum 42 or film 2740. These receive either a separated portion of the epithelium layer 16 or the entire separated epithelium layer 16 in the form of a disk 34 from the separator 14 and then holds the epithelium layer 16 without rupturing the epithelium layer 16 until it is needed to be replaced on the corneal substrate 18. Note that in some embodiments, the holder also preserves—with a hydrating and/or a conditioning substance—a portion of or all of the separated epithelial layer 16, such as in the form of a disk 34 (though other shapes for the separated epithelial layer are possible as well).

FIG. 1A shows an eye 10 of a patient and an epithelial separator device 12. The epithelial separator device 12 includes a separator 14 which can include a separator support 28 and a blunt or dull edge 102 or a wire that is supported by the separator support 28. The separator 14 is structurally designed such that when the blunt edge 102 or wire or other separating structures initially contacts a contact edge of a tissue layer, such as the epithelial layer 16, and when appropriate forces and/or appropriate oscillations, such as 6000 Hz to 15,000 Hz, are applied to the separator, the separator 14 is able to separate (for example by pushing) one or more layers of tissue, such as the layer 16, away from the corneal stroma 1910 while simultaneously not penetrating/cutting into the corneal stroma 1910. Note that the blunt edge 102 initially oscillates side-to-side along direction P shown in FIGS. 1A and 2 with an amplitude ranging from 0.5 mm to 2 mm, more preferably 0.8 mm to 1.6 mm or 1.25 mm to 1.6 mm and most preferably 1.25 mm. It is also possible to oscillate the blunt edge 102 along direct M shown in FIG. 2. In either case, the oscillating blunt edge 102 is simultaneously translated along direction X so that it punctures the epithelial layer 16 until it contacts the corneal substrate 18. Corneal substrate 18 does include Basal membrane 1900 as defined previously. At this point, the blunt edge 102 continues its side-to-side oscillations and translational movement along direction X so that the blunt edge 102 pushes the layer 16 in the manner described above. Note that the separation of the epithelial layer 16 from the corneal substrate 18 is the result of the blunt edge 102 and the separator 14 creating a cleavage plane between two materials with different mechanical properties, namely the epithelial layer 16 and the corneal substrate 18. It is possible for the cleavage plane to be located either 1) between the epithelial layer 16 and the Basal membrane 1900, 2) within the Basal membrane 1900, 3) between the Basal membrane 1900 and the Bowman's layer 1908 and 4) between the Bowman's layer 1908 and the corneal stroma 1910. Without being bound to a particular theory, it is believed that the edge 102 performs separation a section of the corneal substrate 18 that contains solid collagen fibers and applies a distributed force at that point so that the epithelial layer 16 is pushed away from the corneal stroma 1910. Note that the cleavage plane most often occurs in the Basal membrane.

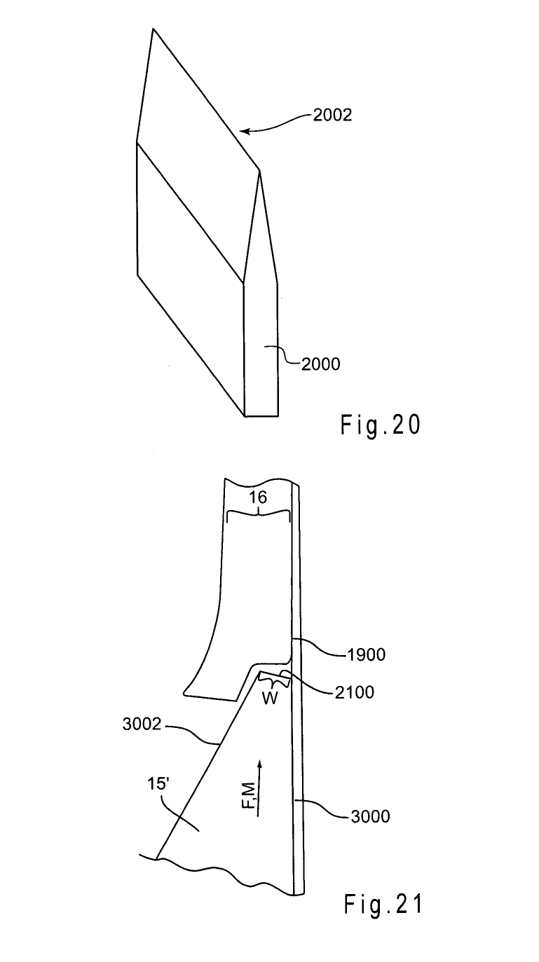

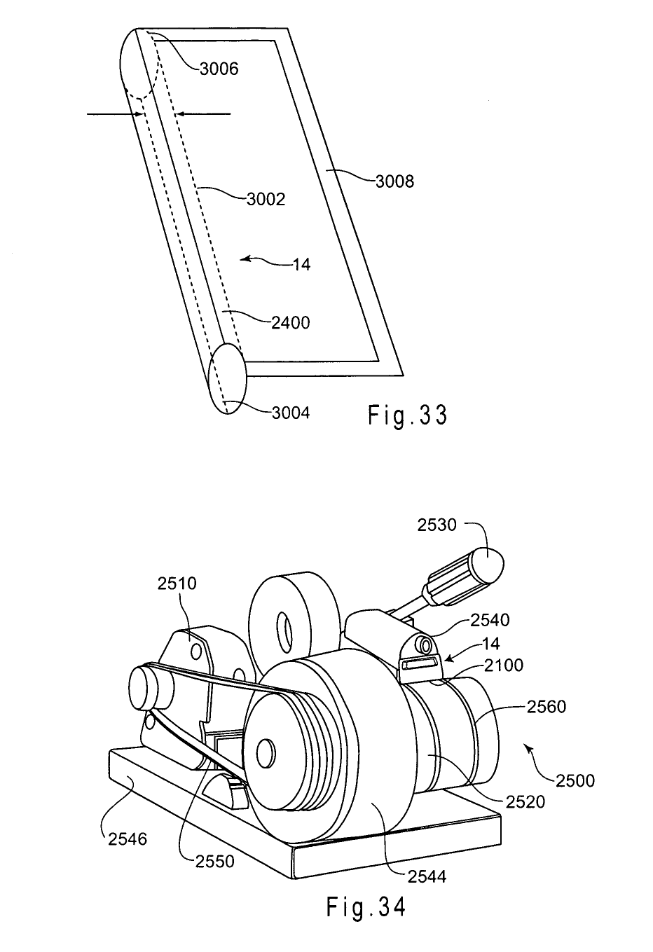

At this point it would be helpful to understand a few concepts as to how the blunt edge 102 interacts with the epithelial layer 16. First, as defined throughout this application, the act of cutting will be defined as the act of separating a physical object into two portions, through the application of an acutely directed force onto a cutting surface of a cutting implement so as generate compressive and shearing forces onto the physical object that cause the physical object to separate along a path defined by the cutting surface. This is in contrast to the previously mentioned puncturing by the blunt edge 102, wherein such puncturing does causes the layer 16 to separate along a path defined by blunt edge 102. Note that the ability for an edge to cut is related to the stress that it creates on an area to be cut through the interaction of the surface of the edge with the area to be cut. Since the stress is inversely proportional to the surface area that applies the force, enlarging the area that is in contact with the corneal stroma 1910 would reduce the stress. Enlarging the area sufficiently will prevent the edge from cutting and so the edge would then qualify as being “blunt” or “dull” as defined in the present application. For example, the prior art “sharp” edge 2002 shown in FIG. 20 can be converted to a “blunt” or “dull” edge 102 by applying the edge 2002 to the machine 2500 shown in FIGS. 33-34 and described later on. In particular, the machine 2500 bends different portions of the initial edge at any one time so that a rounded blunt edge is formed. FIG. 23 gives an example of such bending and shows that the size of the area of the rounded blunt/dull edge 2100″ is greater than the area of the tip of the plate 15′″.

Another possibility for preventing cutting is to lower the force applied to the corneal stroma 1910. In a very general sense, assuming that the applied force is the perpendicular component of the force, F, transmitted by the plate 15 that is oriented at an angle θ, the applied force has a magnitude of Fsinθ. Thus, selecting the angle θ to be sufficiently small, such as zero degrees, would reduce the magnitude of the applied force to such an extent that cutting is prevented. In this situation, the plate 15 could have a rectangular side cross-section wherein a planar base of the plate 15 lies flat on the corneal surface (θ=0°) and the distal end's perpendicular side pushes the layer 16. While the right angled lower corner defined as the intersection of the base and the side is “sharp” it is not angled to apply any cutting force to the corneal stroma 1910. Thus, the right angled lower corner is effectively “blunt” or “dull” with respect to its ability to cut the corneal stroma 1910. Note that in the end the angle is not a critical factor for the plate 15. The key is whether or not the forces applied through the plate 15 are able to overcome the pressure generated by the eye and the resistance to separation generated by the epithelial layer 16. When those forces are overcome by the plate 15, the edge 102 is able to separate the epithelial layer 16 in an effective manner without involving cutting of the corneal stroma 1910. Without being bound by any one theory, it is believed that the separating performed by the present invention involves disrupting the bounding elements or forces that tie distinct and whole entities to one another. Such disrupting results in the distinct entities from no longer being attached to one another while retaining the properties and integrity of the distinct entities.

Using the above analysis, if plate 15 has a trapezoidal-like side cross-section and its base lies flat on the corneal surface and a force is applied to the plate parallel to the base, the angled corner between the base and side of the plate would also be considered “blunt” or “dull” in cutting effectiveness.

During the previously mentioned pushing process performed by the separator device 12, the separator 14, such as via blunt edge 102 pushes the epithelial layer 16 at a so-called contact edge 19 that is the edge of the layer 16 that is presently in contact with the edge 102. As shown in FIGS. 1A, 4, 6, 8 and 9, the contact edge 19 can be linear in shape. The blunt edge 102 pushes the contact edge 19 towards an outer edge 45 of the tissue defining a portion of the outer boundary of the unseparated epithelial layer 16 and located opposite the contact edge 19 as shown in FIGS. 1A, 4, 6, 8 and 9. As shown in FIG. 19, the blunt edge 102 forces the contact edge 19 to be lifted up, separate from corneal stroma 1910 and travel along a portion of the top of the plate 15 as the separator translates along direction X. Note that the previously mentioned top portion of the plate 15 acts as a support surface for the separated epithelial layer 16. The pushing process is such that the blunt edge 102 is incapable of cutting into the corneal stroma 1910 and the tissue layer is substantially intact, when compared to its original state, after the pushing process is complete. Since alcohol or other devitalizing chemicals are not used, the amount of damaged epithelial cells during the process of the present invention is typically less than the amount of damaged epithelial cells that undergo a LASEK procedure.

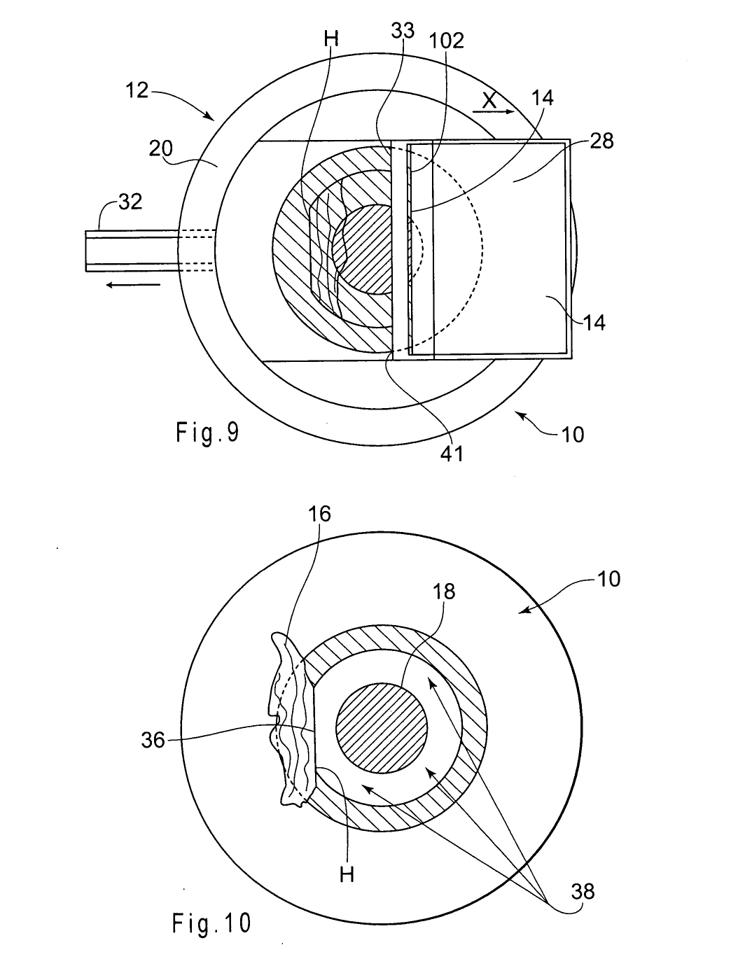

At the end of the pushing process, the separator 14 has either traveled a sufficient distance so as to remove the entire tissue layer or has traveled a lesser distance so that the tissue layer is still adhering to the corneal substrate 18. In the latter case, the separated portion of the epithelial layer 16 is attached to the remainder of the epithelial layer 16 via a hinge H as shown in FIGS. 9-11. As shown in FIGS. 9 and 10, the hinge is linear and is located at the furthest point at which the blunt edge 102 has traveled along the cornea with respect to its initial position prior to initiating separation. Note that for those cases where the final portion of the epithelial layer 16 that is separated is still attached to a hinge H, the final portion typically has a size that has a maximum diameter of about 8.5 mm to 10 mm as measured along a direction parallel to the line defined by the hinge H and a maximum diameter of about 9 mm as measured in a direction perpendicular to the direction parallel to the line defined by the hinge H.

In the case of the separator 14 shown in FIG. 1B, the separator includes a separator support 28 in the form of a rectangular block A. Note that the separator support 28 may be made of a number of rigid and sterile materials, such as metals and plastics. In the case of plastics, the material can be transparent so that the user can see the portion of the eye 10 directly below the separator support 28.

The rectangular block A is made of a material that has sufficient hardness such that it does not substantially deform during the separation process. An example of an acceptable material would be PMMA (Polymethylmethacrylate), ceramic or a well-known surgical instrument metal. The rectangular block A has a height of approximately 3 mm, a length of approximately 12 mm and a width ranging from approximately 2 mm to 6 mm. The bottom surface 47 of rectangular block A has a width w ranging from approximately 2 mm to 6 mm, and a length as measured parallel to the front edge 41 of approximately 12 mm. Note that the bottom of rectangular block A is polished and/or coated with a material, such as parylene, in order to reduce friction between the bottom and the eye. Of course other shapes and orientations for the bottom surface 47 and the block A are possible without departing from the spirit of the invention. For example, it is possible to redesign block A so that the bottom of the block A does not touch the cornea substrate 18.

As shown in FIG. 1B, the separator 14 further includes a planar-like plate 15 that is positioned within an angled recess 17 formed in the block A (see FIG. 1C) of the separator support 28. The recess is preferably dimensioned to have a height/thickness h and width that substantially corresponds to the height/thickness and width of the plate 15 so that the plate 15 is constrained to move along a direction parallel to a plane that contains the recess 17. If movement of the plate 15 in a direction P (see FIGS. 1A and 2) is desired, then the width of the recess can be enlarged by a desired amount.

Note that the plate 15 may be made of a number of substantially rigid and sterile materials, such as polymers, metals and plastics. The plate 15 is designed to be as light as possible while having the properties of avoiding vibration and being able to push the epithelium layer 16 during the separation process. An example of a suitable material is PMMA, wherein in such a case the plate would have a mass of approximately 0.5 g. Note that the plate 15 can be polished and/or coated with a friction reduction material, such as parylene, in order to reduce friction between the plate 15 and the eye

The plate 15 has a thickness ranging from approximately 100 to 400 μm, more preferably 200 to 300 μm and even more preferably 250 μm. The plate has a width of about 10 mm. A blunt edge 102 of the plate 15 has a thickness that can range from the thickness of one half of a single cell of the epithelial layer 16 to the total thickness of layer 16. It is also may be possible to have a thickness that is greater than the total thickness of layer 16. More preferably, the blunt edge 102 of the plate 15 has a thickness between two to three cell layers in thickness. The plate 15 extends at an angle θ that ranges from 0° to 90°, more preferably 10° to 60° and most preferably 20° to 40°. At a distal end of the plate 15, the blunt/dull edge 102 is shaped and designed so that upon being subjected to certain forces and oscillations explained below it will contact one or more layers of the tissue to be removed, such as the epithelial layer 16, without penetrating into the corneal stroma 1910 located below the layers of tissue. In other words, during use of the device 12, the edge 102 is not sharp enough to penetrate into the corneal stroma 1910 so as to cut or excise such tissue during operation of the epithelial separator device 12. Thus, the blunt edge 102 is in direct contrast to the leading edge 2002 of the blade 2000 described previously with respect to FIG. 20 in that edge 2002 is sharp and would risk cutting the corneal stroma 1910 if used to separate layer 16 from the corneal stroma 1910. Note that the blunt edge 102 is that portion of the distal end of the plate 15 that makes contact with the epithelial layer 16 after the initial puncturing through the layer 16 and during the subsequent separation process. The blunt edge 102 also includes adjacent portions of the plate 15 that are in contact with the corneal substrate 18 in a non-cutting manner. Thus, the plate 15 defines an edge that is both blunt with respect to the epithelial layer 16 (because it does not substantially damage the layer 16 during the moving/separation process) and the corneal stroma 1910 (because the surface of the plate 15 contacting the corneal stroma 1910 does not damage the stroma during the separation process).

As shown in FIG. 1A, the separator support 28 and the bottom surface 35 are initially positioned away from the eye of the patient. As shown in FIGS. 1A-B and 2, the separator support 28 of the separator 14 is integrally connected via a throat area B with a very blunt distal end 33 by being very rounded in shape (so as to avoid piercing or cutting the layer 16 during its movement) that serves as an applanator and has a triangular-like cross-section. (Of course, other shapes for the end 33 are possible.)

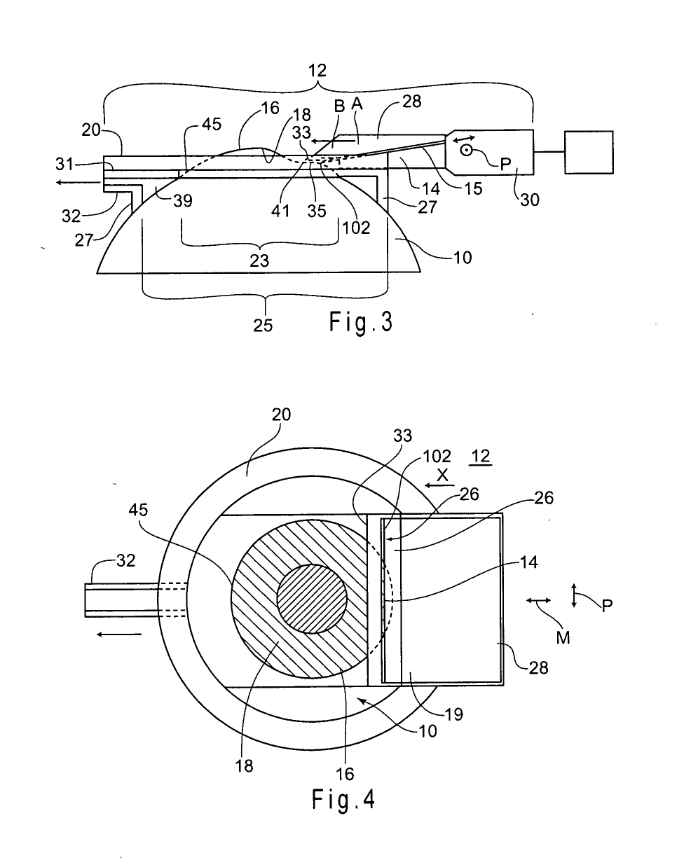

The separator support 28 and the bottom surface 35 are translated along direction X so that the bottom surface 35 makes initial contact with the exterior surface of the eye at a position inwardly of the outer circumferential edge of the epithelial layer 16 as shown in FIG. 3. During such contact, the distal end 33 and the bottom surface 35, acting together as an applanator, compress an exterior surface of the epithelial layer 16 and a corresponding portion of the corneal substrate 18 below it. Such compression causes the layer 16 to be relatively flat from the blunt edge 102 to at least the front edge 41 of the distal end 33, as shown in FIGS. 3, 5 and 7. Thus, the bottom surface 35 of distal end 33 acts as an applanator.

Note that the bottom surface 35 of the distal end 33 needs to be polished as smoothly as possible to decrease the possibility of decreasing the structural integrity of the layer 16 as the surface 35 translates along the top of the layer 16. In addition, a substantially flat bottom surface 35 assists in maintaining a desired angle between the edge 102 and the cornea during separation. The bottom surface 35 may be coated with a friction reduction material, such as parylene, so as to reduce friction between the surface 35 and the eye. The distal end 33 reduces the pressure that is needed to be applied by any post-applinator or block A. Note that in each of the described embodiments herein that applination and/or post-applination or neither can be performed.

The bottom surface 35 of the distal end 33 is substantially horizontal so as to be horizontal with bottom surface 47 (though could have a slight angle) and is elevated and is offset with respect to the blunt edge 102 so that the bottom surface 35 of the end 33 first contacts the outermost cells of the epithelial layer 16 (see FIG. 19). The bottom surface 35 can have a variety of shapes and has a width that extends parallel to the edge 102 and ranges from 1 mm to 10 mm. In particular, the blunt edge 102 is laterally offset from the rear edge of the bottom surface 35 a distance d that is approximately 300 μm and is vertically offset below the bottom surface 35 by a distance ranging from approximately 240 μm to 300 μm also. Note that the lateral offset is a function of how dull the edge is. For example, the duller the edge 102 is, the greater the lateral offset d is. It turns out that the wider the lateral offset d is, the more the edge 102 is pressed against the epithelium/cornea. So if it turns out that the edge 102 is very blunt that it normally would not be able to initially penetrate the epithelial layer 16 at the beginning of the process, the offset d can be increased so as to increase the pressure so as to allow the edge 102 to penetrate the epithelial layer 16. Similarly, the offset d can be decreased if the dullness of the edge 102 is such that only a mild pressure need be applied to the edge 102 to allow it to penetrate the epithelial layer 16.

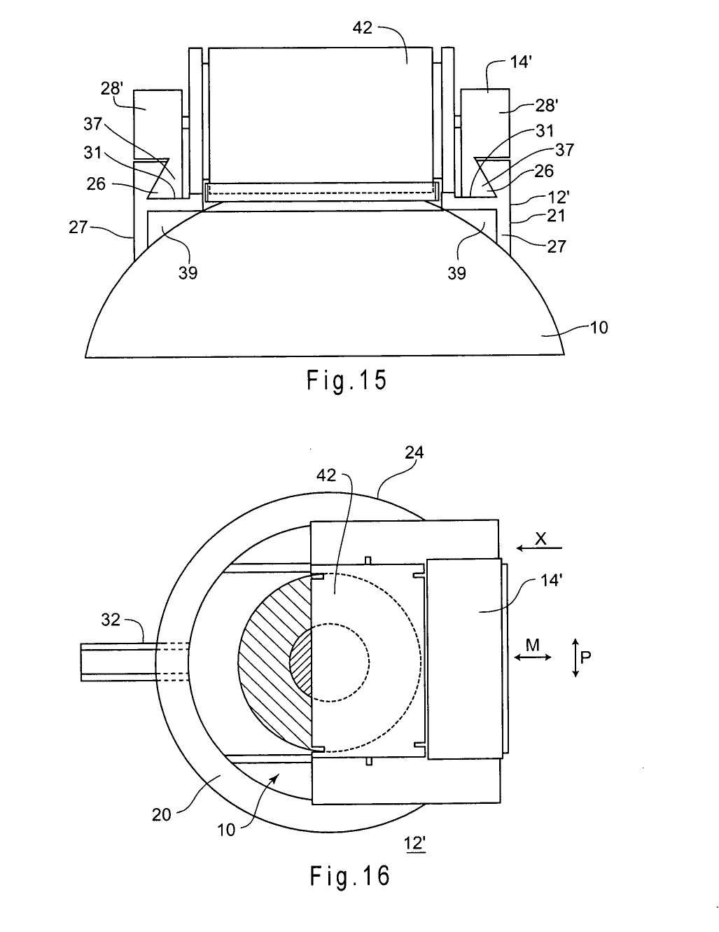

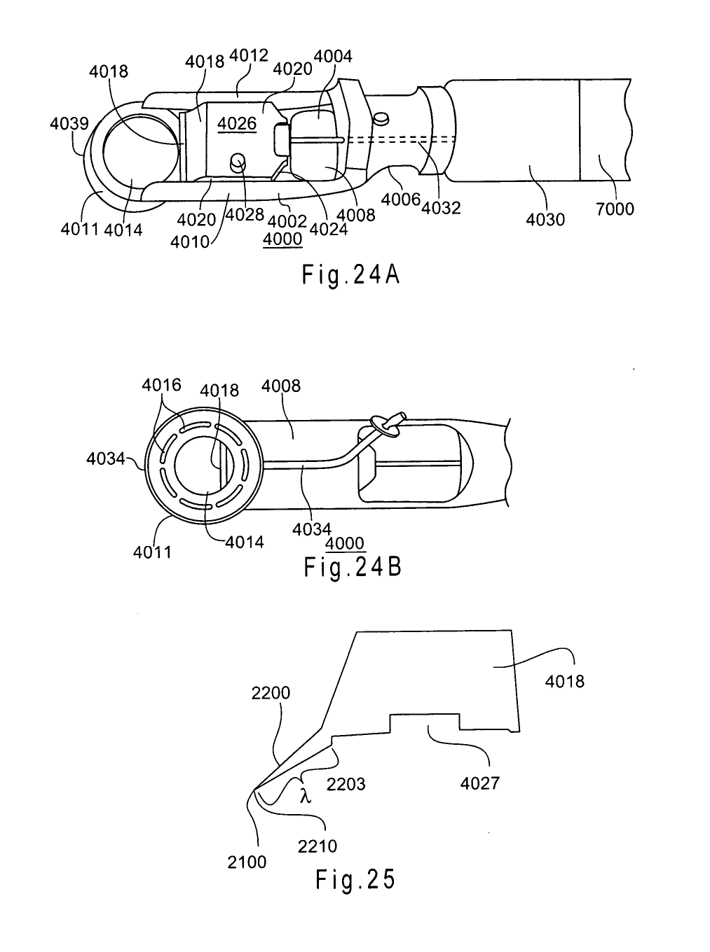

As shown in FIG. 1, the separator device 12 includes an annular ring 20 made of a sterilizable and rigid material, such as a metal including titanium. The ring 20 is supported upon and attached to a housing 21 that defines an upper circular opening 23 and a lower circular opening 25. The upper opening 23 has a diameter of that corresponds to the inner diameter of the ring 20. The bottom opening 25 is defined by an outer wall 27, wherein the bottom opening has a diameter that ranges from 16 mm to 21 mm. The bottom of the outer wall 27 is curved so as to match the external radius of curvature of a portion of eye 10. In the alternative, the outer wall 27 can be slanted instead of being curved depending on the diameter of outer wall 27. The housing 21 is made of a sterilizable and rigid material, such as the material for ring 20, and has a height needed to fit onto the eye and so is based on the diameter of wall 27 and the average anatomic data for the eye. As shown in FIGS. 1A and 2, the bottom of the outer wall 27 is placed on the exterior surface of the eye 10 so that the top surface 31 of the housing 21 is parallel to a limbus of the eye 10. The bottom of outer wall 27 and top surface 31 may be slightly slanted to provide a better fit for certain individual eyes and to avoid having the bottom of outer wall 27 cut into the corneal substrate 18. Consequently, the ring 20 of the epithelial separator device 12 sits on the eye 10 with its plane also substantially parallel to a limbus of the eye 10. As shown in FIG. 2, the ring 20 defines an internal circumference 22 having a diameter ranging from about 10 to about 12 mm and an external circumference 24 that has a diameter ranging from about 13 to about 16 mm and including a groove 26 (best seen in FIG. 15). The groove 26 is dimensioned wider than the diameter of the internal circumference 22 hereinafter called the internal diameter. Male members 37 formed in the rectangular block A of the separator support 28 snugly fit within and slide within the groove 26 to carry the separator 14 on a determined linear path of travel. A similar groove/male member structure is used in the epithelial separator device 400 of FIGS. 24A-B and 25.

As shown in FIGS. 1A-B, the separator support 28 and the plate 15 are coupled to a movement device, such as 1) an oscillation device 30 that applies both a linear and oscillating movement to the edge 102, or 2) a linear movement device. In the case of the oscillation device 30, the device 30 generates a force on the plate 15 along a line of force F that lies within a plane that extends coincident with the plane extending along the length of the recess 17 and the plate 15 so that the plate 15 moves smoothly within the recess 17. The force F is preferably generated via the translational movement of the separator 14 along the direction X caused by the oscillation device 30.

The oscillation device 30 preferably oscillates the plate 15 along direction P shown in FIG. 1A while at the same time translating the plate 15 along the direction X. For such oscillation, the width of the recess to of the block A can be widened by a desired amount and the oscillation device 30 is altered so that the plate 15 can freely oscillate along direction P (as would be readily known to those skilled in art). The frequency of the oscillation along direction P ranges from about 10 Hz to about 10 KHz and the amplitude of such oscillation ranges from 0.5 mm to 2 mm, more preferably 0.8 mm to 1.6 mm or 1.25 mm to 1.6 mm and most preferably 1.25 mm. Such oscillation aids in having the edge 102 separate the layer 16 from the corneal stroma 1910. Note that the oscillation can, instead of direction P or in addition to direction P, be along the direction M.

Electromagnetic or piezoelectric forces on the plate 15 can provide the oscillation, or external rotating or vibrating wires can provide the oscillation. For example, one end of a shaft can be connected to a motor that rotates the shaft about an axis parallel to the shaft itself. A wire is connected to the other end of the shaft and is rotated by the rotating shaft in a manner as used in the microkeratome sold by Refractive Technologies, Inc. of Cleveland, Ohio under the trademark FlapMaker.

In another embodiment, there are two separate motors within the oscillation device 30. That is, the oscillation device 30 is separately coupled to the separator support 28—via a first motor—so as to generate a constant translational velocity for the separator support 28 including the distal end 33 and the blunt end 102; the constant velocity ranging from approximately, 0.8 to 3 mm/s, preferably from approximately 1.2 to 2 mm/s and most preferred approximately 1.5 or 1.6 mm/s towards the center of the eye 10. A second motor, closest to the end of the plate 15, is coupled to the plate 15 so as to oscillate it along direction M. Accordingly, when the first motor translates along direction X, the second motor and the coupled plate 15 (and the entire support 14 coupled to plate 15) translate along direction X. Of course, a separate device can be used to translationally move the support 28 instead of the oscillation device 30.

The net effect of the oscillation and translational movement is that the force F is applied by the edge 102 to the layer 16 along the longitudinal extent of the plate 15 and the recess 17. Ideally, vertical component of the generated force F cancels the upward force generated by the pressure within the eye and the horizontal component of the generated force F cancels the horizontal frictional force generated by the epithelial layer 16. The cancellation of the horizontal components of the force F and the frictional force results in the separator traveling at a constant velocity.

To maintain the ring 20 on the eye 10, for example during oscillation and translation, the housing 21 and the ring 20 fit snugly on the eye 10 so that a seal is formed and the air within the interior cavity 39 is evacuated via circumferential groove 32 positioned on a side of the eye 10. Suction can be applied to the circumferential groove 32 so that the air within cavity 39 is evacuated to ensure stable mounting of the ring 20 to the eye 10. The lower than atmospheric pressures within evacuated cavity 39 range from 300 mm Hg to 700 mm Hg.

FIGS. 3 and 4 are diagrams showing a side and a top view, respectively, of the eye 10 and the separator 14 located in a second position with respect to the eye. This second position represents the time of initial contact between the eye 10 and both the edge 102 and the end 33. At this position, the plate 15 and edge 102 are initially oscillated along direction P and the separator support 28 is moved along direction X so to have edge 102 puncture through the epithelial layer 16. As described previously, as the separator 14 travels to contact the eye 10, a portion of the epithelial layer 16 and the corresponding corneal substrate 18 therebelow is flattened by the applanator defined by the edge 41 and the bottom surface 35 of the end 33. Simultaneously, the blunt edge 102 begins to push the layer 16. Such pushing causes the layer 16 to be pushed toward the center of the eye.

To accommodate the previously mentioned travel of the separator 14 across the cornea, the separator support 28 may have a male member, such as male member 37 of FIG. 15, that engage corresponding bottom grooves, such as grooves 26 of FIG. 15, formed in the ring 20. The separator support 28 is coupled to the device 30 so that the oscillation device 30 pushes the separator support 28 so that the support 28 freely slides in the groove 26. Thus, the plate 15 and edge 33 translate along direction M via oscillation device 30.

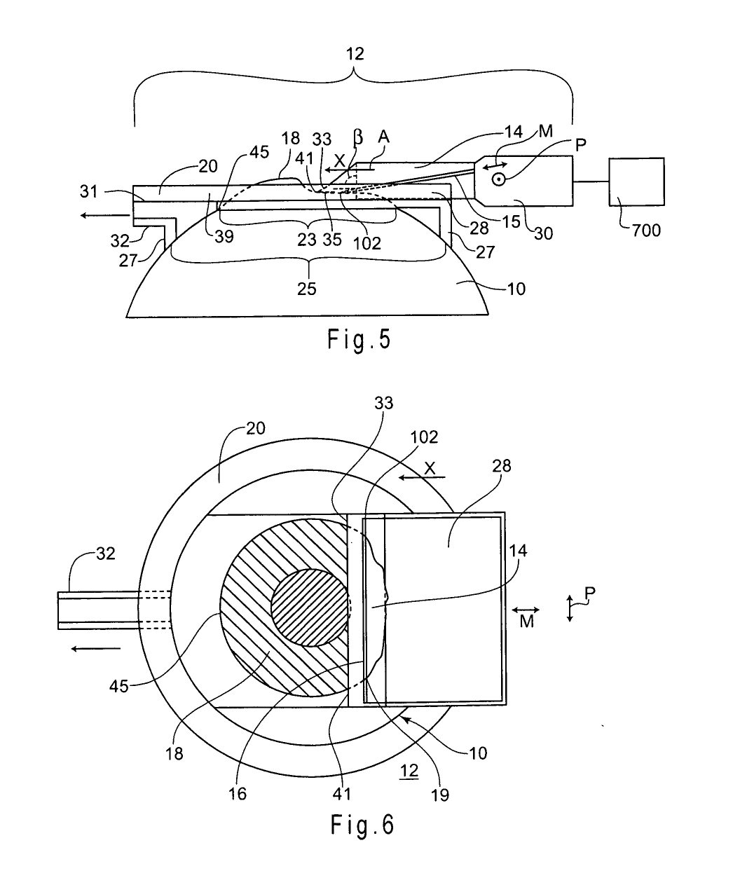

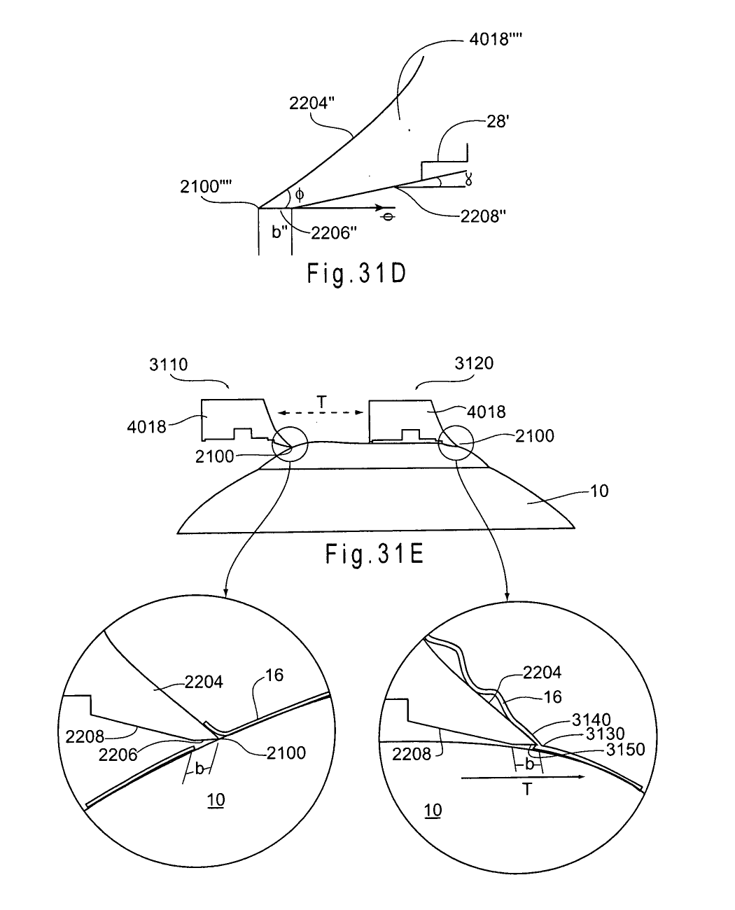

FIGS. 5 and 6 are diagrams showing a side and a top view of the eye 10 and the separator 14 located in a third position. As the separator 14 travels along the cornea 10, the blunt edge 102 of the separator 14 separates the epithelial layer 16 without penetrating or cutting the cornea 18. The edge 41 and the bottom surface 35 of the end 33 continue to applinate a portion of the epithelial layer 16. Further applination is provided by the bottom surface 47 as it contacts the corneal substrate 18 lying below the separated portion of epithelial layer. As the separator 14 travels across the cornea, the applination performed by bottom surface 47 progressively increases due to the fact that more of bottom surface 47 contacts the corneal substrate 18. the epithelial layer 16 is separated from the cornea. This progressive increase in application is believed to cause the interocular pressure to progressively increase. Such an increase in interocular pressure may play a role in the achievement of a complete separation as the increase of intraocular pressure may compensate for the different relationship of angles between the separator and the free cornea at the beginning and at the end of the separation (see FIG. 31E). Without being bound by any particular theory, it is believe that the increased pressure presses the cornea against the edge and prevents the separator from extending through the epithelial layer 16 at the end of the separation process near the end of the eye opposite from the end where the separation process began.

More specifically, FIG. 19 is a diagram representing an enlarged side view of the plate 15 and its blunt edge 102 removing the epithelial layer 16 from the corneal stroma 1910 of the eye 10. The epithelial layer 16 is made up of epithelial cells 1902. The epithelial layer 16 overlies a Basal membrane 1900. The Basal membrane 1900 is formed by basal epithelial cells 1922, the epithelial cells at the base/bottom of the epithelial layer 16. As the basal epithelial cells 1922 grow, so does the Basal membrane 1900. The Basal membrane 1900 is formed from a lamina densa 1904 of about 50 nm in thickness and an underlying lamina lucida 1906 of about 25 nm in thickness. The lamina densa 1906 overlies a Bowman's layer 1908. The epithelial layer 16 anchors to the Bowman's layer 1908 via a complex mesh of anchoring fibrils (type VII collagen) and anchoring plaques (type VI collagen) that interact with the lamina densa 1904 and the collagen fibrils of the Bowman's layer 1908. The Bowman's layer 1908 overlies a corneal stroma 1910.

The epithelial layer 16 is stratified, possessing 5 to 6 layers of epithelial cells 1902. The epithelial layer 16 is typically about 50 to 60 micrometers in thickness. Adjacent epithelial cells 1902 are held together by desmosomes 1912. The epithelial cells 1902 are held to the underlying Basal membrane 1900 by hemidesmosomes 1914 and anchoring filaments. A bottom surface of the epithelial layer 16 includes numerous microvilli and microplicae, i.e., ridges, whose glycocalyx coat interacts with, and helps to stabilize, a precorneal tear film. New epithelial cells 1902 are derived from mitotic activity in the Basal membrane 1900 layer. New epithelial cells 1902 displace existing cells both superficially and centripetally.

As described above, the plate 15 includes a blunt leading edge 102 to push the epithelial cells 1902 as the plate 15 and blunt edge 102 move under the epithelial layer 16. The plate 15 preferably pushes the epithelial cells 1902 and does not exert a force that could disrupt the intercellular bonds such as the desmosomes 1912. Accordingly, the plate 15 is able to separate the epithelial layer 16 substantially in one piece without cutting the corneal stroma 1910 so that it can be transferred back onto its original area of rest upon the tissue that remains once the laser ablation process is finished as will be described below. Note that the point of separating the epithelial layer 16 has been found to often occur at the border between the lamina densa 1904 and the lamina lucida 1906. In other instances, the Basal membrane 1900 and/or some debris or portion from the Bowman's layer 1908 may remain attached to the separated portion of the epithelial layer 16. In yet further instances, a portion of the epithelial layer having a thickness less than the total thickness of the epithelial layer can be removed. However, as described previously such partial removal is not desirable in performing the present invention. The plate 15 preferably pushes the bottom two to three layers of epithelial cells 1902 which probably contain a majority of the shear strength of the epithelial layer 16.

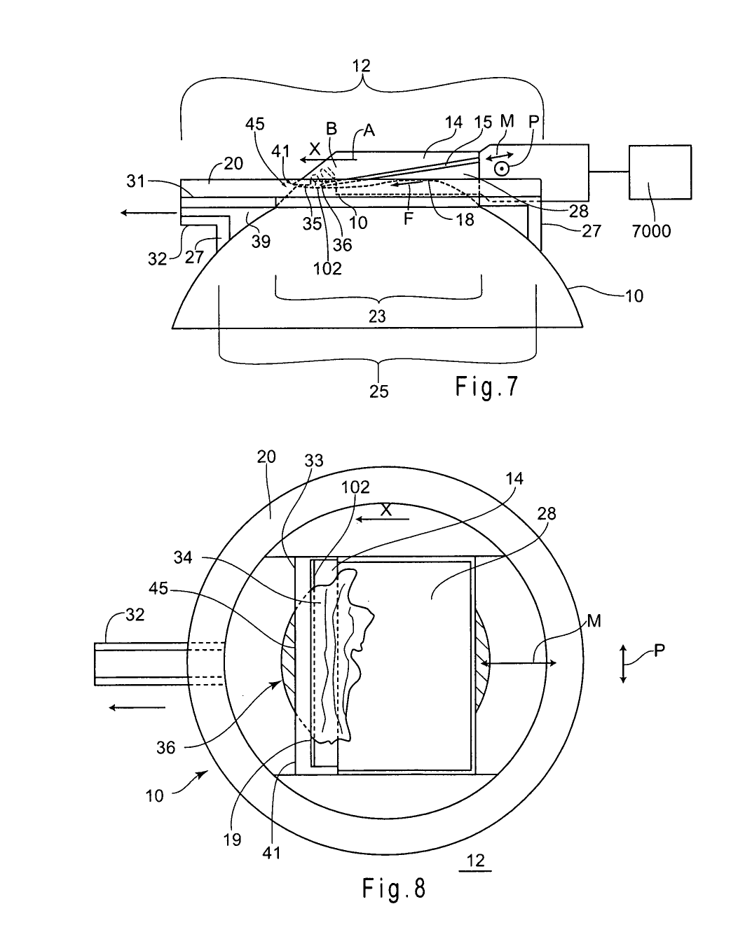

FIGS. 7 and 8 are diagrams showing a side and a top view of the eye 10 and the separator 14 located in a fourth position. In one embodiment, the travel of the separator 14 is controlled so that a circular-like area of the separated epithelial layer 16 is formed that is concentric with either the limbus or the pupil (pupil is not generally in the center of the cornea; in many eyes there can be a slight eccentricity). The movement of the separator 14 and its blunt edge 102 is programmed so that the blunt edge 102 stops at a desired position, such as just prior to forming layer 16 in the shape of a circle. Such stoppage creates a hinged area between the portion of the layer 16 that has been separated and the portion of the layer 16 that has not been separated from corneal substrate 18. At this stage, the separated portion of the layer 16 has the shape of a “D”, wherein of course the straight portion of the “D” is much smaller than the height of the “D.” For example, during the final phase of the gradual movement shown in FIGS. 1-8, the separator 14 and the blunt edge 102 stop movement at a point slightly before complete separation of a circular disk so that a D-shaped epithelial disk 34 is formed that is attached to a hinge located at an edge 36 of the layer 16 located at a left portion of the eye as shown in FIG. 8. In another embodiment, the epithelial disk 34 is completely detached from the corneal stroma 1910, for example, as described below. Note that either the D-shaped epithelial disk 34 or the completely detached disk can be shaped so as to form a pocket so as to receive a lens.

Note that the above removal process can be done in combination with a handheld spatula that lifts/manipulates the layer 16. The handheld spatula is similar to the one used during the previously described LASEK procedure. In addition, the entire removal process is mechanical in that no chemicals, such as alcohol, are used to interact with and loosen the epithelial layer 16. It should be noted that a couple of saline drops may by applied to the separator device such that the saline drops interact with the edge 102 and the eye so that the layer does not stick to surface 2204 during the separation process. The saline drops in no way interact with the epithelial layer 16 so as to loosen the bond between the epithelial layer 16 and the corneal substrate 18.

FIG. 9 is a diagram showing a top view of the eye 10 and the separator 14 located in a retracted position after the epithelial disk 34 as been formed. In this embodiment of the separator 14, the applanator and blunt edge 102 are formed at a higher position relative to the surface 31 of FIG. 1A than the applanator and blunt edge 102 of FIGS. 1-8. Thus, the applanator and the blunt edge 14 contact the eye along planes that are located closer to the apex of the eye. Thus, the cross-sectional areas of the eye intersected by the higher planes are smaller in area than those formed by lower positioned planes. Thus, a smaller epithelial disk 36 is separated when the applanator and the edge are formed at a higher position. Note that the separation diameter is denoted to mean the diameter of the portion of the corneal substrate 18 that is denuded of epithelial cells. Thus, in the example given above, the separation diameter equals the diameter of the generally circular-shaped area corresponding to the intersection of the corneal substrate 18 and the plane defined by the blunt edge 102 in the direction of movement.

After the separator 14 is retracted, suction to the circumferential groove 32 is turned off and the epithelial separator device 12 is removed from the eye 10. Referring also to FIG. 10, after the epithelial separator device 12 is removed, a deepithelialized area 38 is exposed that substantially corresponds to a shape and size of the area that the separator 14 and edge 33 contacted during travel. At this point where the epithelial layer is separated from the surface of the cornea, the surgeon is able to perform surgery on the exposed area. For example, the surgeon can center a laser ablation applied to the area 38 according to the laser manufacturer.

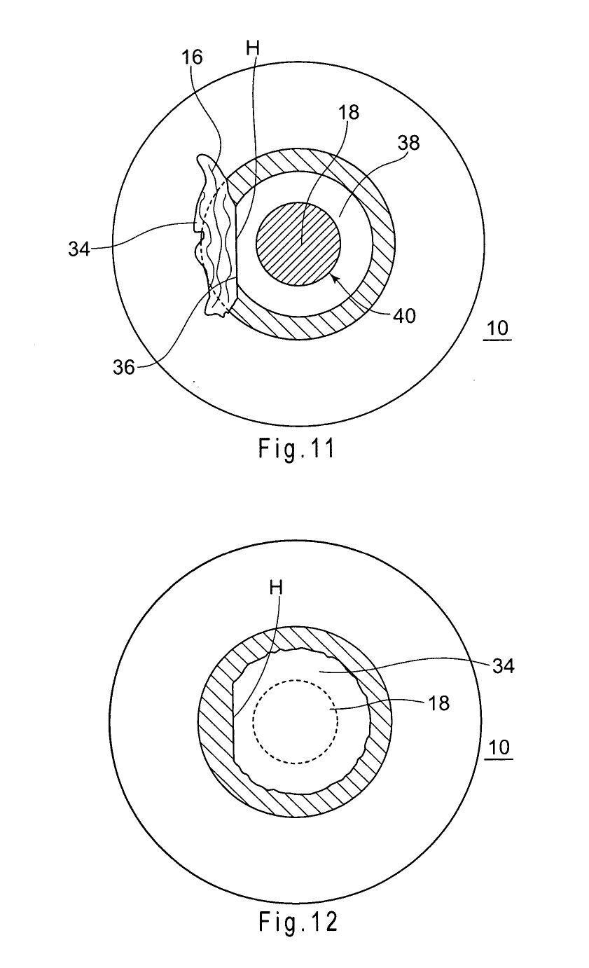

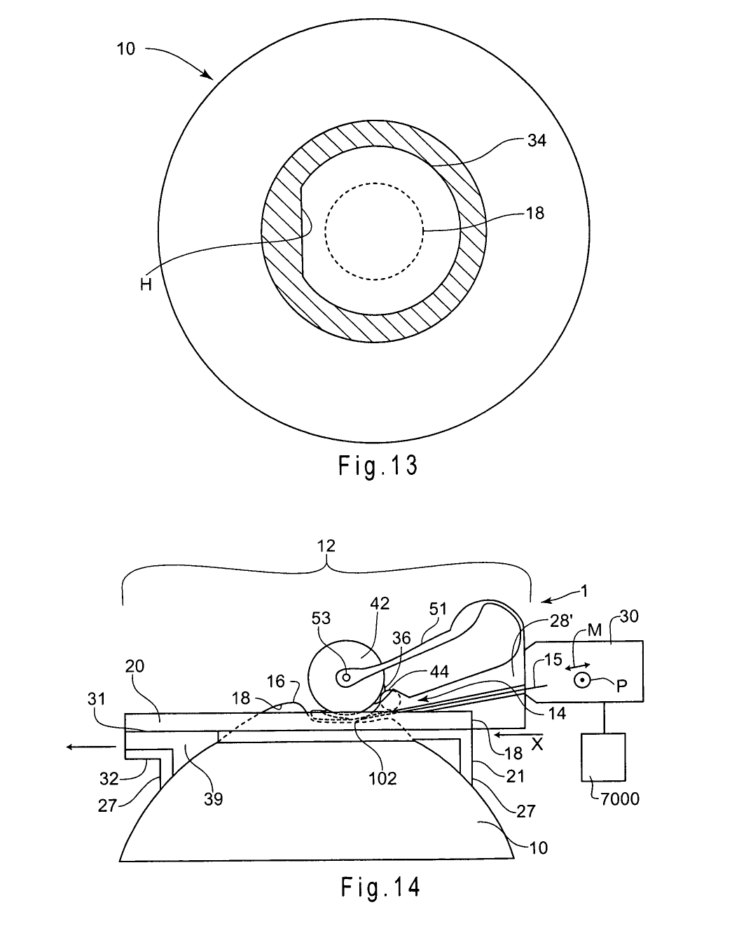

FIG. 11 shows a top view of the eye 10 of FIG. 10 after laser ablation is performed on the deepithelialized area 38. The laser ablation forms an irradiated area 40 on the eye 10. Referring to FIG. 12, thereafter, the epithelium disk 34 is replaced on the remaining portions of the corneal substrate 18 of the eye 10 to aid in the healing process. Note that replacing the epithelium disk 34 can be performed by using a well known spatula that is commonly used to manipulate a LASIK flap. The spatula may also be used to lift and/or manipulate the epithelial layer 16 during the separation process. Referring to FIG. 13, once replaced on the remaining portions of the corneal substrate 18, the epithelium disk 34 is preferably smoothly stretched into place via such well known surgical instruments as manipulators, spatulas, forceps, or a sponge. The macroscopic integrity of the epithelium disk 34 allows the epithelium disk 34 to be easily manipulated into place. Since the epithelial layer does not adhere to the cornea, a soft contact lens is placed over the epithelial layer and on the eye as in PRK so as to act as a patch. The replaced epithelium disk 34 allows for a more pain free recovery when compared with the case when the ablated portion of the eye 10 is left exposed as in PRK, because exposed nerves of the eye 10 are covered by the epithelium disk 34. New epithelium cells grow under the replaced epithelium disk 34 as the cells of the epithelium disk 34 die. The separated epithelial disk 34 is typically totally replaced by new cells in about 3-6 days.

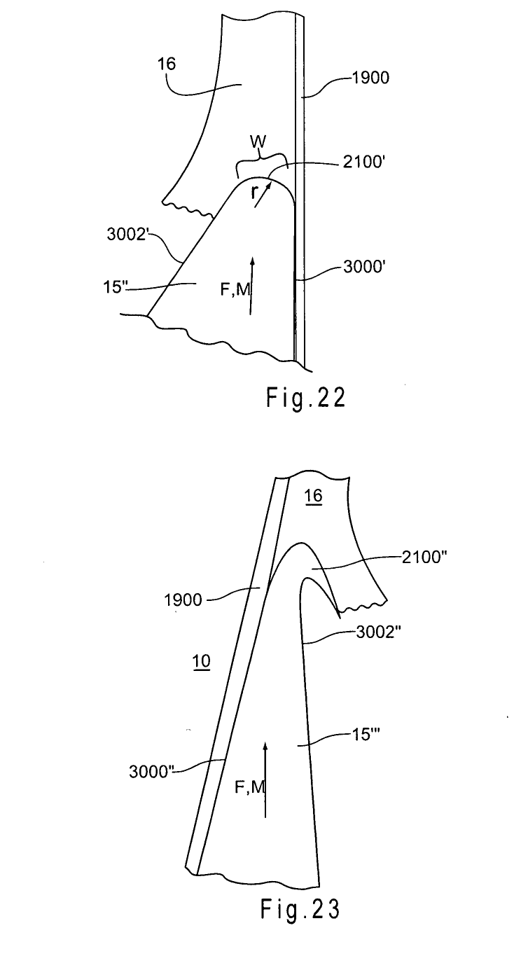

FIG. 21 is a diagram showing a side view of an embodiment of a leading edge 2100 that can be used with a plate 15′. The plate 15′ is similar in structure with the plate 15 of FIGS. 1-9, except that it uses the leading edge 2100. The plate 15′ is supported by separator support 28 so that the plate 15′ and separator support 28 define another embodiment of a separator to replace the separator 14 of FIG. 1. The plate 15′ is moved by oscillation device 30 in the same manner as plate 15 as described previously with respect to FIGS. 1-9. During the removal of the epithelial layer 16, the bottom planar surface 3000 of the plate 15′ is positioned adjacent and parallel to the cornea underlying the Basal membrane 1900 while the flat leading edge 2100 contacts the layer 16. Such an orientation may be used in order to avoid cutting of the corneal stroma 1910. Accordingly, the planar leading edge 2100, the top surface 3002 and the bottom surface 3000 can be treated together as defining a blunt edge with respect to the epithelial layer 16 and the stroma since the epithelial layer 16 is moved without substantial damage and the stroma is not damaged during movement of the bottom surface 3000 and leading edge 2100. The bottom surface 3000 acts as an applanator as the leading edge 2100 moves to remove/separate a portion of the layer 16. The top surface 3002 acts as a support surface upon which the separated epithelial layer 16 can lie during the separation process. The leading edge 2100 of the separator 14 should not have a width, w, that is too large such that it will increase the chance that the edge 2100 penetrates the epithelial layer 16. The leading edge 2100 preferably includes a width w ranging from 5 to 25 micrometers, and more preferably a width w that is about 15 micrometers, though it can be greater than 50 micrometers. Note that the embodiment of FIG. 21 is advantageous over the embodiment of FIG. 22 since there is no need to round the end of the separator.

FIG. 22 is a diagram showing a side view of a second embodiment of a leading edge 2100′ that can be used with a plate 15″. The plate 15″ is similar in structure with the plate 15 of FIGS. 1-9, except that it uses the leading edge 2100′ to separate the portion of the epithelial layer. The plate 15″ is supported by separator support 28 so that the plate 15″ and separator support 28 define another embodiment of a separator to replace the separator 14 of FIG. 1. The plate 15″ is moved by oscillation device 30 in the same manner as plate 15 as described previously with respect to FIGS. 1-9. During the removal of the epithelial layer 16, the bottom planar surface 3000′ of the plate 15″ is positioned adjacent and substantially parallel to the cornea underlying the Basal membrane 1900 while the leading edge 2100′ contacts the layer 16. Accordingly, the leading edge 2100′, the top surface 3002′ and the bottom surface 3000′ can be treated together as defining a blunt edge with respect to the epithelial layer 16 and the stroma since the epithelial layer 16 is moved without substantial damage and the stroma is not damaged during movement of the bottom surface 3000′ and leading edge 2100′. The leading edge 2100′ is rounded instead of flat. The leading edge 2100′ has a width w similar to that of the leading edge 2100 of FIG. 21. The leading edge 2100′ has a radius of curvature, r, having a value that ranges from 1 micron to 20 microns or about one half the thickness of the edge 2100′.

The bottom surface 3000′ acts as a post-applanator as the leading edge 2100′ moves to remove the layer 16. Note that the terms “post-applanator” and “post-applinate” as used for the present embodiment and other embodiments described herein regard the situation where the separator includes a surface that applanates the remaining portion of the corneal stroma 18 that is present after the edge has separated the epithelial layer 16. Such a post-applanator provides several advantages. For example, the post-applanator is embodied as a single component and so it has no significant tolerances in positioning with respect to the eye when compared with a multi-piece assembly. A second advantage is that the post-applanator is in contact with the remaining portion of the corneal substrate 18 not the epithelium therefore ensuring a constant relationship between the edge 2100′ and the remaining portion of the corneal substrate 18. This second advantage is evident in eyes that not have previously undergone a surgical treatment. In particular, the variation in epithelial layer thicknesses from one patient to another is greater than the variation in the cornea dimensions of the same patients. Such variations in epithelial thickness can result in the pre-applinator affecting the positioning of the edge 2100′ while the post-applanator would have little effect on the positioning of the edge 2100′. The above-described process and embodiment provide a safe separation process that can be easily repeated in a consistent manner.

Note that the planar surface 3000′ may be angled in a similar manner as shown in FIG. 1A. In either the flat orientation of FIG. 22 or the angled orientation, the forces applied through the plate 15″ are able to overcome the pressure generated by the eye and the resistance to separation generated by the epithelial layer 16.

FIG. 23 is a diagram showing a side view of a third embodiment of a leading edge 2100″ that can be used with a plate 15′″. The plate 15′″ is similar in structure with the plate 15 of FIGS. 1-9, except that it uses the leading edge 2100″. The plate 15′″ is supported by separator support 28 so that the plate 15′″ and separator support 28 define another embodiment of a separator to replace the separator 14 of FIG. 1. The plate 15′″ is moved by oscillation device 30 in the same manner as plate 15 as described previously with respect to FIGS. 1-9. During the removal of the epithelial layer 16, the bottom planar surface 3000″ of the plate 15′″ is positioned adjacent and parallel to the cornea underlying the Basal membrane 1900 while the leading edge 2100″ contacts the layer 16. Such an orientation may be used in order to avoid cutting of the corneal stroma 1910. Accordingly, the leading edge 2100″ and the bottom surface 3000″ can be treated together as defining a blunt edge with respect to the epithelial layer 16 and the stroma since the epithelial layer 16 is moved without substantial damage and the stroma is not damaged during movement of the bottom surface 3000″ and leading edge 2100″. The leading edge 2100″ is constructed, for example, by bending the leading edge 2002 of the blade 2000 shown in FIG. 20. The leading edge 2100″ preferably includes a diameter of about 5 to 25 micrometers, or a radius between about 2 to 13 micrometers, and more preferably includes a diameter of 15 micrometers. The bottom surface 3000″ acts as a post-applanator as the leading edge 2100″ moves to remove the layer 16. The top surface 3002″ acts as a support surface upon which the separated epithelial layer 16 can lie during the separation process. Note that the leading edge and bottom surface can be angled with respect to the corneal stroma.

FIGS. 24A-B and 25 show a second embodiment of an epithelial separator device 4000. The epithelial separator device 4000 includes a housing 4002 made of a rigid material, such as a metal. The housing 4002 defines a track 4004 and a drive coupling 4006 integrally attached to one another. The track 4004 is defined by a planar surface 4008 and two vertical side walls 4010 and 4012 that are spaced from each other by a distance of approximately 12 mm to 16 mm, more preferably 13 mm to 15 mm and most preferably 14 mm. At one end of the track 4004 a circular opening 4014 having a diameter of approximately 11 mm is formed in the surface 4008. On the bottom of the surface 4008, a 19 mm diameter annular skirt 4011 is formed that is centered about the opening 4014. The skirt 4011 is made of the same material as the surface 4008 and integrally attached thereto. Within the skirt 4011 are seven arc-shaped protrusions 4016 as shown in FIG. 23B that extend from the bottom of the surface 4008. The protrusions 4016 are of equal length, are equi-angularly spaced from one another and are centered about the opening 4014. Each of the protrusions 4016 has a beveled free end that is curved so as to match the external radius of curvature of a portion of eye 10 when contacting the cornea. The protrusions 4016 perform the functions of 1) keeping the conjuctiva in place and 2) uniformly distributing the vacuum in the circumference of the skirt 4011. The conjuctiva is tissue like a membrane that covers the eye at the portion where the skirt 4011 attaches to. The conjuctiva sometimes may be somehow loose and therefore rise when vacuum is applied within skirt 4011. If it rises in the suction ring it can go to the suction port (where tube 4034 goes into the suction ring) and block it. In that case the vacuum would be not distributed evenly around the suction ring and the whole device would not be stable on the eye.

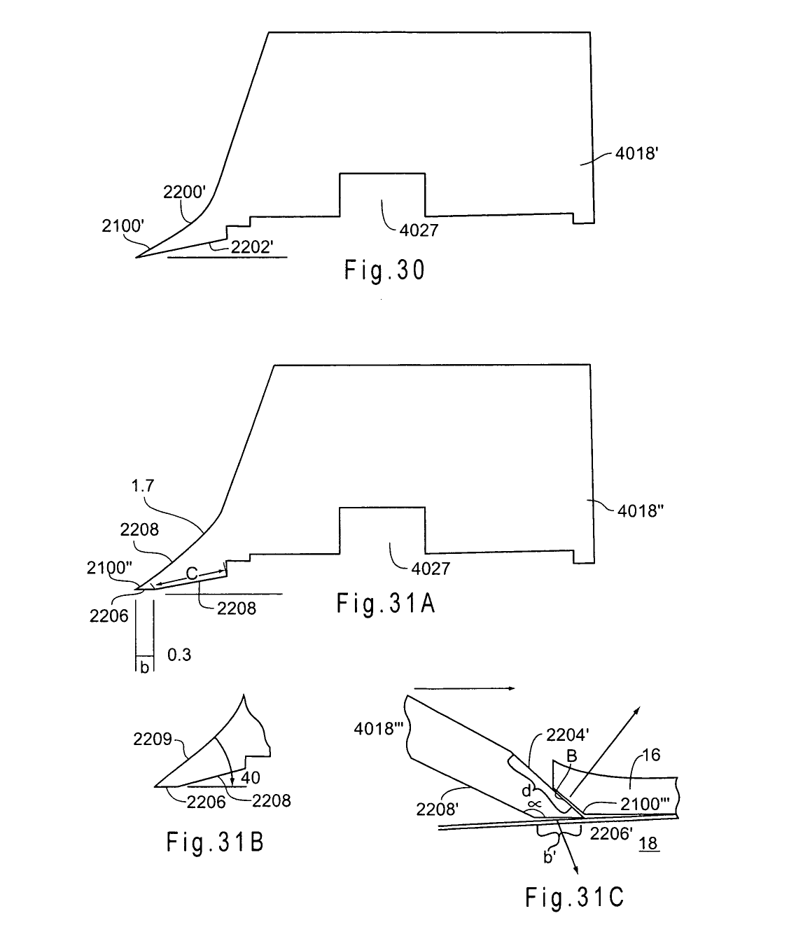

The device 4000 includes a separator 4018 as shown in FIGS. 24-25. Other separators can be used in device 4000 instead of separator 4018. For example, the separator 14 of FIGS. 1-9, the separator support 28 containing the plates 15′, 15″ and 15′″ of FIGS. 21-23 and the separators 14′ and 14″ of FIGS. 30-31 can be used in device 4000. As shown in FIG. 25, the separator 4018 includes a blunt leading edge 2100′″ formed at the end of planar surfaces 2200 and 2210. The planar surfaces 2200 and 2202 are angularly separated from one another by an amount ranging from 0 to 90 degrees, preferably 10 to 60 degrees, preferably approximately 10 to 30 degrees. The bottom planar surface 2202 is oriented in a range from 20 to 60 degrees, preferably approximately 26 degrees with respect to the planar bottom surface 2210 (width along direction of movement approximately 300 μm) and has a length, d, of approximately 1.5 mm. The leading edge 2100′″ pushes the layer 16 while the bottom surface 2210 acts as an applanator and flattens the eye while the separator 14 moves. Since the leading edge 2100′″ and surfaces 2200, 2202 and 2210 do not substantially damage the epithelial layer 16 and do not damage or cut the stroma during the separation process, the leading edge 2100′″, surface 2210 and at least portions of surfaces 2200 and 2202 can be viewed jointly as a blunt edge. In addition, the surface 2200 can act as a support surface for supporting the separated epithelial layer during the separation process.

The separator 4018 is positioned within a metallic holder 4020 that includes: 1) a planar support surface 4022 that slides upon surface 4008, 2) a U shaped side wall 4024 and 3) a pivotable top surface 4026. The support surface 4022 includes parallel bottom side male members, similar to male member 37 of FIG. 15, that engage corresponding bottom grooves formed in the side walls 4010, 4012, similar to groove 26 of FIG. 15, After the top surface 4026 is pivoted to an open position, the separator 4018 is slid into the cavity defined by the holder 4020 so that the rectangular recess 4027 is snugly engaged by rectangular male member (not shown) formed on the top portion of surface 4022 and a leading edge of the separator 4018 extends out of the holder 4020. Once properly positioned, the top surface 4026 is pivoted to a closed position wherein the top surface 4026 lies just above the top surface of the separator 4018. Next, the separator 4018 is held in place upon tightening a screw 4028 of the top surface 4026 that threadingly engages an opening formed in the body of the holder 4020 located below the closed top surface 4026. Such a screwing action causes the bottom surface of the surface 4026 to engage with the top surface of the separator 4018 and so trapping the separator 4018 within the holder 4020.

The separator 4018 is designed to contact and remove an epithelial layer 16 without making an incision in corneal tissue located below the epithelium. The separator 4018 removes an epithelial layer 16 located above a corneal stroma 1910 of the eye 10 in a manner similar to that described previously with respect to separator 14 with respect to FIGS. 1-9. The separator 4018 is not sharp enough to excise corneal tissue during operation of the epithelial separator device 4000.

As shown in FIG. 24A, the separator 4018 is coupled to an oscillation device 4030 via a rod 4032 that has one of its ends attached to the rear of the holder 4020. The other end of the rod 4032 passes through an opening formed in the drive coupling 4006 and is directly coupled to the oscillation device 4030. The other end of the rod 4032 defines a stop that limits the distance that the rod 4032 can be translated relative to the coupling 4006. When the stop engages the portions of the drive coupling 4006 that define the opening, the leading edge of the separator 4018 just passes past the opening nearest the end 4034 of the device 4000. The oscillation device 4030 generates a force on the separator 4018 parallel to the surface 4008.

In operation, the skirt 4011 and the protrusions 4016 are placed on the exterior surface of the eye 10 so as to form a seal and so that the surface 4008 is parallel to a limbus of the eye 10. To maintain the skirt 4011 on the eye 10, for example during oscillation, air within the interior cavity is evacuated via a portal 4034 positioned on a side of the eye 10. Suction can be applied via portal 4034 so that the air within cavity 39 is evacuated to ensure stable mounting of the skirt 4011 to the eye 10.

Note that a device similar to device 4000 is described in U.S. Patent Application Publication No. 2005/0055041 A1, the entire contents of which are incorporated herein by reference.

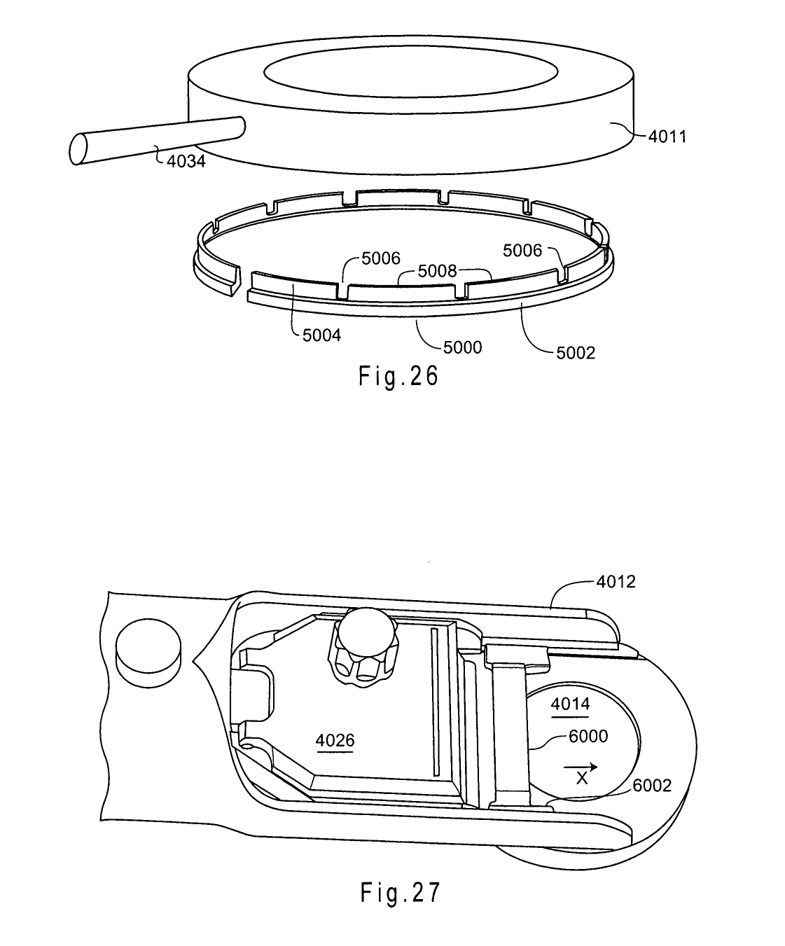

Note that it is known that applying suction can result in tissue, such as conjunctiva tissue, being displaced from the eye so as to plug up the portal 4034. To overcome this, a C-shaped guard 5000 is used as shown in FIG. 26. The guard 5000 is made of a resilient material, such as a sterilizable metal or hard plastic, and has a diameter that is slightly larger than that of the interior of the skirt 4011. Thus, when the guard 5000 is placed within the cavity defined by skirt 4011, the bottom 5002 of the guard 5000 engages the bottom portion of skirt 4011 in a spring-like manner so as to create a seal. The top portion 5004 of the guard is annular like in shape and is slightly recessed inward with respect to the exterior of the bottom 5002. The top portion 5004 has several rectangular openings 5006 formed which define a plurality of arc-like guards 5008. When the guard 5000 is inserted within the skirt 4010, one of the guards 5008 is positioned in front of the portal 4034. In operation, the guard 5000 allows suction to be performed via the passages defined by the recessed top portion 5004, the skirt 4011 and the openings 5006. The guards 5008 intercept/block tissue before it can clog up the portal 4034. Note that the guard 5000 can be used with the device of FIG. 1 in a similar manner.

Once the device 4000 is positioned upon the eye, the oscillation device 4030 is operated in a manner similar to that described previously with respect to the oscillation device 30 so as to provide translational motion and vibration to the separator 4018. Furthermore, the epithelial layer 16 is removed in a manner similar to that described previously with respect to the device of FIGS. 3-8 without cutting the cornea 18. Note that while applanation of the cornea 10 is performed by the separator 4018 alone, it is possible to perform applanation in a serial manner with the separator 4018 by employing a metal applanator 6000 that is attached to and spaced in front of the holder 4020 as shown in FIGS. 27-29. Accordingly, in operation the cornea is first applanated by applanator 6000 and then post-applanated by the separator 4018 while the portion of the layer 16 is being separated/removed in the manner described previously.

Note that in all of the embodiments discussed previously and subsequently, separation of the epithelium layer 16 can still be accomplished without using pre- and/or post-applination.

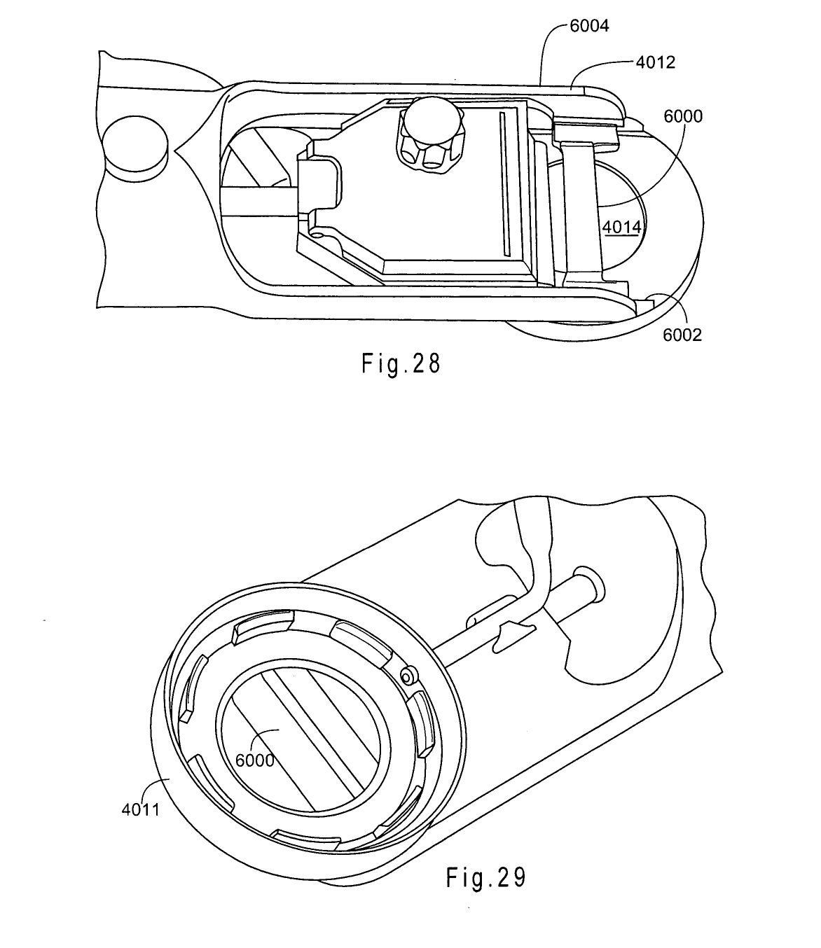

The applanator 6000 has a width of about 2 mm as measured along the direction X. As shown in FIG. 29, the lower portion of the applanator 6000 is rounded and polished. As shown in FIGS. 27 and 28, the applanator 6000 is in the shape of an “H” with the vertical parts 6002 being bent so that ends of the parts 6002 fit in and slide within the previously mentioned bottom grooves of the side walls 4010, 4012. Note that the male members 6004 of the support surface 4022 extend towards the parts 6002 so that when metal holder 4020 moves forward, the male members engage the vertical parts 6002 and push the applanator 6000 as well. However, when the support surface 4022 is retracted in the opposite direction, the applanator 6000 does not move and so is not retracted.

In operation, the cornea is first applanated by applanator 6000 and then post-applanated by the separator 4018 while the layer 16 is being removed in the manner described previously. The applanator flattens the eye before the blunt edge reaches the eye. Note that applanator 6000 can be adapted to be used with the device shown in FIG. 1 so as to operate in a similar manner as described above. As mentioned previously, such post-applination provides the advantages of not having significant tolerances when compared with a multi-piece assembly and having little effect on the positioning of the edge 2100′.

After the layer 16 is removed, the separator 4018 is retracted in a manner similar to that shown in FIG. 9. After the separator 4018 is retracted, suction is turned off and the epithelial separator device 4000 is removed from the eye 10. After the epithelial separator device 4000 is removed, a deepithelialized area 38, such as shown in FIG. 10, is exposed that corresponds to a shape and size of the area that the separator 4018 contacted during travel.

Next, laser ablation of the exposed area is performed so as to form an irradiated area 40 on the eye 10 as shown in FIG. 11. Referring to FIG. 12, thereafter, the epithelium disk 34 is replaced on the remaining portion of the corneal substrate 18 of the eye 10 to aid in the healing process. Referring to FIG. 13, once replaced on the remaining portion of the corneal substrate 18, the epithelium disk 34 is preferably smoothly stretched into place. Note that the disk 34 can be removed using the devices described previously with respect to FIGS. 14-18.

FIG. 30 shows a side view of a second embodiment of a separator 4018′ that can take the place of the separator 4018 (see FIG. 25) in the epithelial separator device 4000 of FIGS. 24A-B. In particular, the separator 4018′ can be slid into the cavity defined by the holder 4020 so that the rectangular recess 4027 is snugly engaged by rectangular male member (not shown) formed on the top portion of surface 4022 and a leading edge 2100′ of the separator 4018′ extends outward. It is also envisioned that the separator 4018′ can take the place of the separator 14 and the end 33 of the epithelial device 12 of FIGS. 1-9. The blunt leading edge 2100′ of separator 4018′ is rounded and formed at the ends of planar surfaces 2200′ and 2202′ that are angularly separated from one another by an amount ranging from 0 to 90 degrees, preferably 10 to 60 degrees, most preferably approximately 10 to 30 degrees. The bottom surface 2202′ is oriented approximately 20 to 60 degrees, preferably 26 degrees, with respect to the top surface 31 of the housing 21 and has a length, d, of approximately 1.5 mm. The leading edge 2100′ pushes the layer 16 while the bottom surface 2202′ acts as an applanator and flattens the eye while the separator 14 moves. Since the leading edge 2100′ and surfaces 2200′ and 2202′ do not substantially damage the epithelial layer 16 and do not damage or cut the stroma during the separation process, the leading edge 2100′″ and at least portions of surfaces 2200′ and 2202′ can be viewed jointly as a blunt edge. In addition, the surface 2200′ can act as a support surface for supporting the separated epithelial layer during the separation process.

FIGS. 31A-B show side views of a third embodiment of a separator 4018″ that can take the place of the separator 4018 (see FIG. 25) in the epithelial separator device 4000 of FIGS. 24A-B. In particular, the separator 4018″ is slid into the cavity defined by the holder 4020 so that the rectangular recess 4027 is snugly engaged by rectangular male member (not shown) formed on the top portion of surface 4022 and a leading edge 2100″ of the separator 4018 extends outward. It is also envisioned that the separator 4018″ can take the place of the separator 14 of the epithelial device 14 of FIGS. 1-9. The embodiment is disclosed in a U.S. Provisional Patent Application Ser. No. 60/599,368, filed on Aug. 6, 2004, the entire contents of which are incorporated herein by reference. The blunt leading edge 2100″ of separator 4018″ is formed at the end of planar surfaces 2204 and 2206 that are angularly separated from one another by an amount ranging from 0 to 90 degrees and more preferably 30 to 60 degrees, more preferably approximately 40 degrees. The bottom surface 2206 has a length, b, ranging from 0 to 0.4 mm, preferably approximately 0.3 mm or 0.15 mm and is oriented parallel to surface 31 as shown in FIG. 1A. Note that has b is decreased in size, the amount of pressure applied to the eye via surface 2206 increases. The bottom surface 2206 is oriented 20 to 40 degrees, preferably approximately 26 degrees, with respect to an angled bottom surface 2208, which has a length, c, of approximately 1.2 mm. The leading edge 2100″ pushes the layer 16 while the bottom surface 2206 acts as an applanator and flattens the eye while the separator 14 moves. Note that bottom surface 2206 enhances applanation when compared with when the blunt edges of separators of FIGS. 25 and 30 are used for applanation. Note that angling the bottom surface 2206 allows for a proper pressure to be applied to the eye by surface 2206, allows for proper orientation of the edge and reduces the amount of rubbing between the cornea and surface 2206. Since the leading edge 2100″ and surfaces 2204, 2206 and 2208 do not substantially damage the epithelial layer 16 and do not damage or cut the stroma during the separation process, the leading edge 2100″, surface 2206 and at least portions of surfaces 2204 and 2208 can be viewed jointly as a blunt edge. In addition, the surface 2204 can act as a support surface for supporting the separated epithelial layer during the separation process.

A variation of the separator 4018′″ is shown in FIG. 31C, wherein the distal end of the separator 4018′″ that includes surfaces 2204, 2206 and 2208 is replaced by a separator 4018″″ that is similar to separator 4018′″ except that its distal end material has a trapezoidal-like side cross-sectional shape. Note that the separator 4018 may have its edges rounded. The material includes surfaces 2204′, 2206′ and 2208′ that define leading edge 2100′″. Surface 2206′ has a length b′ of about 0 to 400 μm, preferably 50 μm to 350 μm, and more preferably 150 or 300 μm. Since the leading edge 2100′″ and surfaces 2204′, 2206′ and 2208′ do not substantially damage the epithelial layer 16 and do not damage or cut the stroma during the separation process, the leading edge 2100′″, surface 2206′ and at least a portion of surface 2204′ can be viewed jointly as a blunt edge. In addition, the surface 2204′ can act as a support surface for supporting the separated epithelial layer during the separation process.

FIG. 31D shows a variation of the separator 4018 as separator 4018″″. The separator 4018″″ includes surfaces 2204″, 2206″ and 2208″ that define leading edge 2100″″. Surface 2206″ has a length b″ of about 0 to 400 μm, preferably 50 μm to 350 μm, and more preferably 150 or 300 μm. Since the leading edge 2100″″ and surfaces 2204″, 2206″ and 2208″ do not substantially damage the epithelial layer 16 and do not damage or cut the stroma during the separation process, the leading edge 2100″″, surface 2206″ and at least a portion of surface 2204″ can be viewed jointly as a blunt edge. In addition, the surface 2204″ can act as a support surface for supporting the separated epithelial layer during the separation process. The top surface 2204″ is oriented at an angle Φ of about 0 to 90 degrees, preferably 30 to 60 degrees and most preferably 40 degrees with respect to the bottom surface 2206″. The bottom surface 2206″ is oriented at an angle θ of about minus twenty to thirty degrees, preferably minus 10 to 10 degrees, most preferably zero degrees with respect to horizontal, such as with respect to the separator support 28′ of the separator 4018″″. The angled bottom surface 2208″ is oriented at an angle γ of about zero to forty degrees with respect to horizontal. Once again, since the leading edge 2100″″ and surfaces 2204″, 2206″ and 2208″ do not substantially damage the epithelial layer 16 and do not damage or cut the stroma during the separation process, the leading edge 2100″″, surface 2206″ and at least a portion of surface 2204″ can be viewed jointly as a blunt edge. In addition, the surface 2204″ can act as a support surface for supporting the separated epithelial layer during the separation process.

FIG. 31E shows separators 4018, 4018′, 4018″, 4018′″ and 4018″″ with their respective leading edges 2100, 2100′, 2100″, 2100′″, 2100″″ as they travel along T across the eye 10. The separators 4018, 4018′, 4018″, 4018″″ and 4018″″ may travel from a start position 3110 to an end position 3120, and back again. As they travel, the separators 4018, 4018′, 4018″, 4018′″ and 4018″″ are able to separate the epithelial layer 16 from the cornea of the eye 10. The surfaces 2206, 2206′ of the separators 4018, 4018′, 4018″, 4018′″ and 4018″″ may act to applanate or flatten the surface of the eye 10. The pressure to the eye 10 may increase as the separators 4018, 4018′, 4018″, 4018′″ and 4018″″ move from the start position 3110 to the end position 3120 because more of the eye 10 is flattened when the separators 4018, 4018′, 4018″, 4018′″ and 4018″″ reach the end position 3120 than at the start position 3110. When the separators 4018, 4018′, 4018″, 4018′″ and 4018″″ reach the end position 3120 of travel, a hinge 3130 may remain where a removed portion 3140 of the epithelial layer 16 connects with a portion 3150 that is still attached to the eye 10. The length b of the surface 2206 may be varied depending on a desired thickness or robustness of a hinge 3130 to remain. A smaller rather than bigger length b may minimize the splitting of the removed portion 3140 from the attached portion 3150 so that a thicker hinge 3130 may remain.

Note that the leading edges 2100, 2100′, 2100″, 2100′″ and 2100″″ of FIGS. 25 and 30-31 are formed from separators 4018, 4018′, 4018″, 4018′″ and 4018″″, respectively, that are made of a number of rigid and sterilizable materials, such as metals and plastics. Of course other materials are possible. The lines of intersection between two connected surfaces can be dulled by placing the separators in a container containing glass beads, wherein the container is rotated so that the tumbling of the glass beads lessens the sharpness of the lines of intersection. The separators 4018, 4018′, 4018″, 4018′″ and 4018″″ with their respective leading edges 2100, 2100′, 2100″, 2100′″, 2100″″ are able to separate the epithelial layer 16 substantially in one piece without cutting the cornea so that it can be transferred back onto its original area of rest upon the tissue that remains after the laser ablation process is finished as will be described below. Note that Bowman's layer 1908 may be removed once the laser ablation process is finished as will be described below.

The edge 2100′″″, as well as other leading edges 2100, 2100′, 2100″, 2100′″, 2100″″, can be textured to make rough the edges 2100, 2100′, 2100″, 2100′″, 2100″″, 2100′″″. The texturing procedure can be controlled to provide a rough texture and/or waviness to the edge. One possible procedure is to machine wavy lines before polishing and applying controlled polishing to shine the surface but not fully remove the waviness. Other patterns and/or random texturing may be used, such as a dimple pattern. A surface that is too shiny may not remove the epithelial layer 16 with as much consistency as a surface that includes at least some roughness. The rough texture can be used with other separators such as the separators of the embodiment of FIGS. 1-9, FIGS. 14-16, FIG. 19, FIGS. 21-25, FIGS. 27-31, FIG. 33 and FIGS. 36-37.

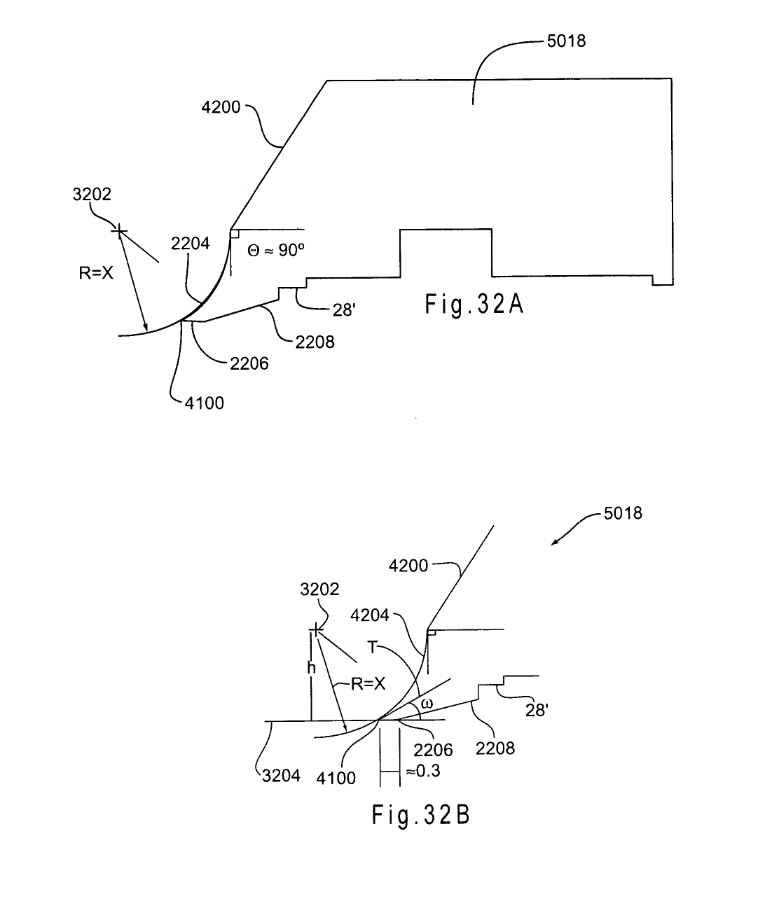

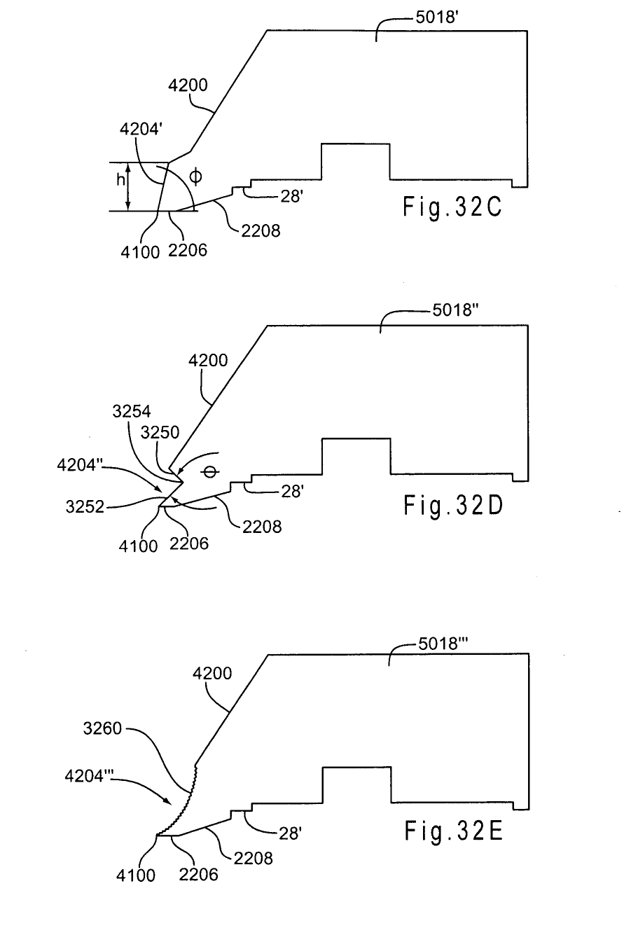

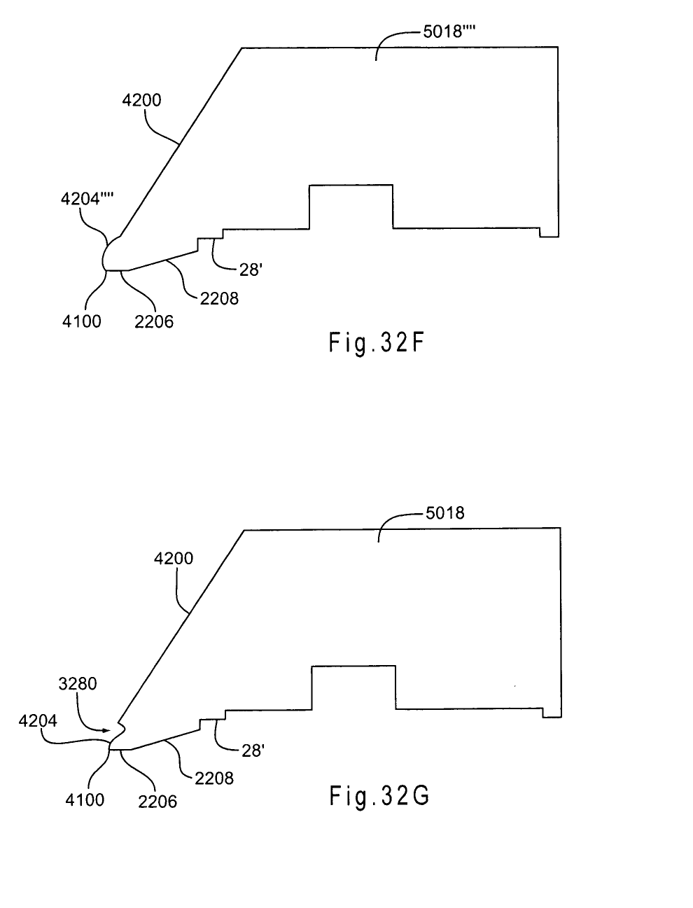

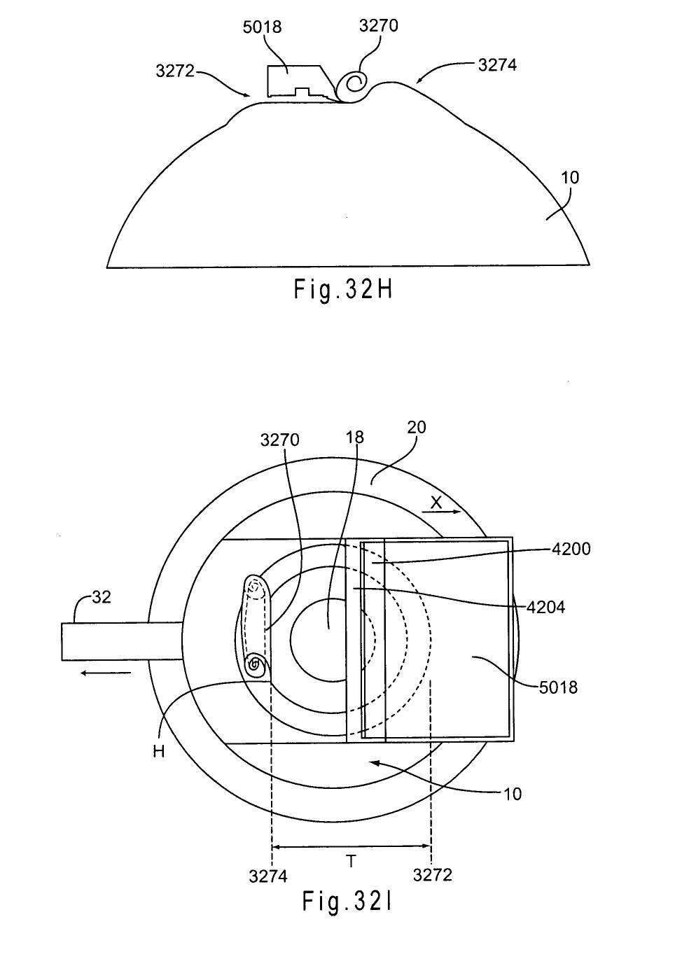

FIGS. 32A-G show side views of a various embodiments of a separator 5018 that can take the place of the separator 4018 (see FIG. 25) in the epithelial separator device 4000 of FIGS. 24A-B. The separators of FIGS. 32A-G can replace other separators such as separator device 12 (FIGS. 1-9) and other separators such as the separators shown in FIGS. 14-16, FIG. 19, FIGS. 21-25, FIGS. 27-31, FIG. 33 and FIGS. 36-37. The separators of FIGS. 32A-G may include an upper surface that includes a structure adapted to roll a separated portion, such as the epithelium, of the eye 10.

In the embodiments of FIGS. 32A-B, a top surface 4204 of the separator 5018 is curved in the general shape of a plow or a semi-cylinder. The surface has a cross-sectional shape that substantially coincides with an arc or a curve. The arc may substantially coincide with a portion of a circle having a radius R of about 0.3 mm to 2 mm, more preferably 0.5 to 1.5 mm and more preferably 0.8 to 1.4 mm. The curved shape may substantially coincide with shapes other than a circle, such as an ellipse, parabola, hyperbola or other conic shapes. The curved shape may also include other shapes, such as a logarithmic shape, a curve with progressive change in curvature such as an Archimede's spiral (see http://mathworld.wolfram.com/ArchimedesSpiral.html) or a combination of all previous curves joined by third order spline polynomials. The curve begins at leading edge 4100 and continues until reaching a top angled surface 4200. The top angled surface 4200 begins when the angle theta (θ) between a line tangential to the curved surface 4204 and a line parallel with respect to support surface 28′ is about ninety degrees. In one embodiment, the distance h from center of curvature 3202 to the horizontal plane 3204 defined by surface 2206 is less than approximately one radius R. The distance h may be implemented to produce an angle ω of a tangential plane T (see FIG. 32B) to the horizontal plane 3204 of approximately 40 degrees. The tangential plane T is the tangent of the curved surface 4204 at the leading edge 4100. For any given radius of curvature, the height h can be varied so that the angle ω varies from 0 to 90 degrees more preferably 15 to 60 degrees more preferably 30 to 40 degrees.

The curved surface 4204 can act to roll the epithelial layer as the layer is separated from the eye 10. The layer can roll away from the direction of the movement of the separator 5018 or towards the direction of movement of the separator 5018, depending on a shape of the surface 4204. A length of the curved top surface 4204 may vary such that there is enough of a surface area to roll the epithelial layer onto itself. As the edge 4100 strikes the eye 10, the edge 4100 penetrates the epithelium, without penetrating the cornea stoma. As the edge 4100 moves across the eye 10 at least a portion of the epithelial layer, which covers the eye 10, is removed. The removed portion of the epithelial layer includes one or more layers of the epithelial cells. The epithelial layer can be completely removed from the eye 10 or remain attached to the eye 10, such as at an end hinged in a manner as described previously. After the separator has traveled a sufficient distance across the cornea to separate a desired portion of the epithelial layer, the separator 5018 is retracted. During retraction, the rolled epithelial layer may remain in the place last pushed on the eye 10 without traveling back with the separator 5018. As shown in FIG. 32I, the rolled epithelial layer remains at a position such that it does not obscure the area of the stroma to be ablated by the laser. The rolled epithelial layer may remain attached to the hinge H. Alternatively, the rolled epithelial layer may be detached and/or removed from the eye 10 so that no hinge exists. If the epithelial layer accidentally travels back with the separator 5018, the surgeon may manually push the epithelial layer to a final position shown in FIG. 32I so as to expose the stroma. The rolled epithelial layer may be advantageous because a surgeon need not further handle the epithelial layer before ablating the eye 10, such as with a laser. If the surgeon needs to handle the epithelial layer, the rolled layer may be easier to maneuver than a flap. If the epithelial layer is not rolled, the surgeon may need to push the epithelial layer aside before ablation. Preferably, no pre-applanator spaced from the edge 4100 is used that could unroll the rolled epithelial layer when the separator 5018 is retracted. The edge 4100 can perform post-applanation. Moreover, an applanator may be used that stays at the end of travel of the edge 4100 and does not move with the edge 4100 during retraction. Separators, such as separators 15′, 15″, 15′″, 4018, 4018′, 4018″, 4018′″, 4018″″, and 2400 (FIGS. 21, 22, 23, 25, 30, 31A-D and 33) can be adapted to include the general plow shape. Note that while the above examples regard a dull edge being used, a sharp edge can be used as well if the edge is to perform a cutting function such as that used with the LASIK process. If an applanator is used, the applanator can be shaped, positioned and/or arranged such that the rolled layer does not rest on the applanator. Alternatively, the applanator can be positioned such that the rolled layer gathers and rests on the applanator, and moves with the applanator, such as when the separator 5018 and applanator are being moved forward or backwards across the eye.

FIGS. 32C-G are side views according to yet other embodiments of the separator. In FIG. 32C, a surface 4204′ may include a flat portion with a height h of about 0.5 mm (500 microns) to about 2 mm (2000 microns), and more preferably about 1 mm (1000 microns). The flat surface 4204′ may be positioned at an angle Φ from about 20 degrees to about 90 degrees, and more preferably 40 degrees or more, with respect to the surface parallel to the axis of travel along the eye 10, such as a plane parallel to the support surface 28. The height and the angle Φ may be chosen such that a removed epithelial layer may roll onto itself. As the removed epithelial layer travels up along surface 4204′, and the epithelial travels high enough along surface 4204′, the weight of the epithelial layer and gravity force a free end of the epithelial layer drop in front of the separator 5018′. As separator 5018′ moves across the eye 10, the epithelial layer travels up along surface 4204′ and then back down to roll onto itself. Once initially rolled onto itself, surface 4204′ pushes the rolled-up surface so as to roll the surface across the eye 10, such as like rolling a carpet.

In the embodiment shown in FIG. 32D, the separator 5018″ may include an upper planar surface 3250 and a lower planar surface 3252 that form a side-wise V-shape such as >. An acute angle θ defined between the two planar surfaces 3250, 3252 ranges from about 0 to about 180 degrees. The distance from the separating edge 4100 and the point of intersection 3254 of the two planar surfaces is a distance that ranges from 15 microns to 4 mm. As an epithelial layer is separated, it moves up the lower surface 3252 and past the point of intersection 3254. As the epithelial layer moves past the point on intersection 3254, the upper surface 3250 contacts the layer and may induce the separated epithelial layer to roll onto itself. Thereafter, the rolled layer may continue to be rolled as the separator 5018″ moves across the eye 10, such as described in FIG. 32C.

Also, it is envisioned, such as in the embodiment of the separator 5018′″ of FIG. 32E, that the curved surface 4204′″ includes surfaces other than a smooth curve, such as a surface 4204′″ that includes multiple steps 3260. The steps of the surface 4204′″ may rise up in a generally curved manner. The steps 3260 may include generally symmetrical shapes or may vary such that some steps are longer and or higher than others, depending on an implementation. The steps 3260 may have substantially straight edges and corners, and/or at least a portion of the steps may include generally rounded edges or corners.

In the embodiment of the separator 5018″″ of FIG. 32F, the curved surface 4204″″ may be convex, instead of concave, so that the removed epithelial layer may roll up in an opposite manner than would happen for the separator of FIGS. 32A-B. The shape of the surface 4204″″ may jut towards or away from the epithelial layer to be contacted. As the epithelial layer moves up along the surface 4204′″, the weight of the epithelial layer and gravity may act to force the epithelial layer to roll onto itself. Thereafter, the rolled layer may continue to be rolled as the separator 5018″″ moves across the eye 10, such as described in FIG. 32C.

In embodiment of FIG. 32G, any of the curved surfaces of FIGS. 32A-F may also include a lip portion 3280 to help ensure that the removed epithelial layer rolls onto itself.

FIG. 32H is a side view of the separator of any of FIGS. 32A-32G or any other separator that acts to roll the epithelial layer 3270 as the separator 5018, 5018′, 5018″, 5018′″, 5018″″ moves across the eye 10. As shown in FIG. 32H, the separator is about half way along its path of travel. Separation of the epithelial layer 3270 starts in a first position 3272 and ends in a second position 3274, different from the first position 3272. The epithelial layer 3270 may remain at or near the second position 3274 without needing to be manipulated as the separator is retracted. Due to its shape, the rolled epithelial may tend not to return with the separator.

FIG. 32I is a top view of the separators of any of FIGS. 32A-32G or any other separator that acts to roll the epithelial layer 3270 as the separator 5018, 5018′, 5018″, 5018′″, 5018″″ moves across the eye 10. Separation of the epithelial layer 3270 starts in a first position 3272 and ends in a second position 3274, such as at hinge 11. The epithelial layer 3270 may remain at or near the second position 3274 without needing to be manipulated as the separator 4018′″″ is retracted. Due to its coiled shape, the rolled epithelial may tend not to return with the separator 5018, 5018′, 5018″, 5018′″, 5018″″ when retracted. The position of the rolled epithelial layer 3270 may be substantially different from the first position 3272, such that the position is at least half the total distance traveled T by the separator 5018, 5018′, 5018″, 5018′″, 5018″″ in one direction.

FIG. 33 is a diagram showing a perspective view of a wire 2400 that could be used as a replacement for the separators 14, 4018, 4018′, 4018″ and 4018′″ according to a preferred embodiment. The wire 2400 includes a generally elliptical or circular cross-sectional shape. The wire 2400 includes a leading edge 3002 with a width of about 5 to 25 micrometers. The wire 2400 is preferably manufactured from a material that is strong enough to push the epithelium without breaking. Exemplary wire materials include titanium and its alloys, tungsten and its alloys, steel alloys and carbon fibers. The two ends 3004 and 3006 of the wire 2400 are preferably attached to a yoke 3008 that is coupled to the oscillation device 14. The yoke 3008 maintains tension in the wire 2400 so that the leading edge 3002 remains relatively straight while it is pushing the epithelial layer 16.

Note that in all of the embodiments of the separators shown in FIGS. 1-33, the separators and associated oscillation devices are positioned so that they move the separators towards the bridge B of the nose of the patient (see FIG. 2). This movement causes the epithelial flap to be positioned on the bridge of the nose. Such a position can lead to damage to the flap should the patient blink his or her eyes.

As an alternative, the separators and oscillation devices can be rotated by 90 degrees so that the separators move towards the eyebrow of the patient. In this case, the epithelial flap would be positioned in the more advantageous position on the eyebrow E of the patient (see FIG. 2). The oscillation devices of FIGS. 1-33 may contact the cheekbone of the patient which could hinder positioning the ring on the eye. This can be corrected by either making the suction ring deeper or redesigning the oscillation device housing structure so that it avoids the cheek.

Note that in all of the embodiments of the separators shown in FIGS. 1-33, the oscillation devices can be altered to be controlled by a closed loop control system 7000. Such a control system would be designed so as to control the distance traveled by the separator along the direction X. The control system can control the moving device, such as the oscillator 30, via a stop mechanism. Such stop mechanism can either be an electronic control that is electrically connected to the moving device and sends signals to the moving device to ensure that the separator does not travel past a predetermined distance. The stop mechanism can also be embodied as a stop that is positioned in the path of the moving separator and when contacted by the separator, the separator is prevented from further movement so that the separator is prevented from traveling the predetermined distance. In addition, the control system 7000 would control the velocity of the separator along direction X so that the velocity is constant during the entire separation process, even when the separator contacts the stroma and epithelial layer. The control system 7000 would also control the frequency of oscillations along direction M or P so that the frequency is constant during the entire separation process, even when the separator contacts the stroma and epithelial layer.

FIG. 14 is a diagram showing a side view of the eye 10 and an embodiment of an epithelial separator device 12′ that includes a rotating drum 42. The device 12′ essentially combines the structure of the epithelial separator device 12 of FIGS. 1-9 with a drum structure that will be discussed below. In particular, the device 12′ includes a U-shaped yoke 51 that supports the drum 42 therebetween via an axle 53. The axle 53 can be supported by both legs of the yoke 51, like a bicycle wheel, or by just one of the legs, like a paint roller. To rotate the drum 42, the epithelial separator device 12′ may include a rotating gear 44. The gear 44 could also be used to provide movement to the separator support 28′ that is similar to the separator support 28 shown in FIG. 1B. The separator 28′ is similar in structure to separator 28 of FIG. 1, except that it includes the yoke 51.

Referring also to FIGS. 15 and 16, front and top views, respectively, of the epithelial separator device 12′, the rotating gears 44 could be symmetrically placed on both sides of the separator support 28′ of the separator 14′. The oscillating device 30 can provide for rotation of the gears 44 and the gears 44 can travel on rails, for example toothed rails, which run parallel to the groove 26. In addition, the drum 42 acts as an applanator as shown in FIG. 14. A second applanator, similar to applanator 6000 of FIGS. 27-29 can be used in device 12′ so as to be positioned prior and in series with the drum 42.

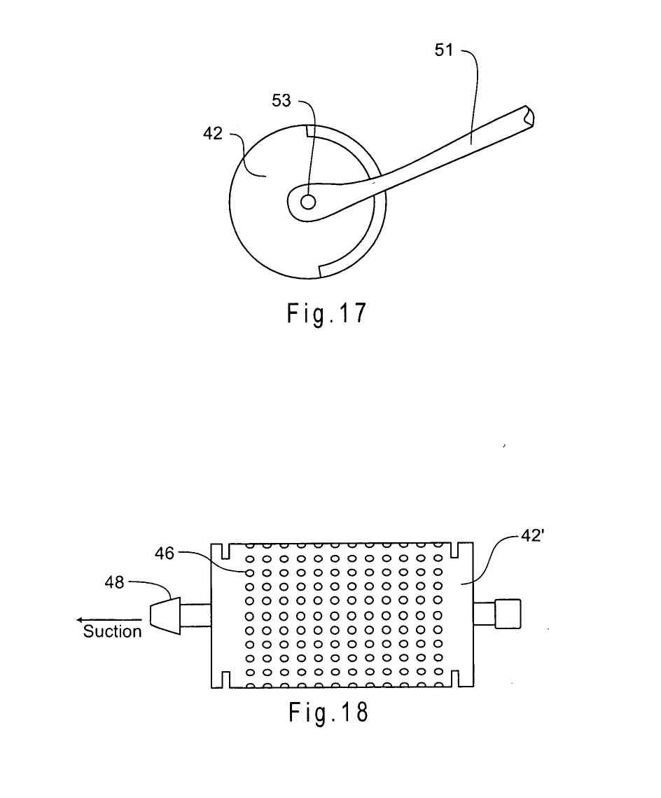

Since a typical thickness of an epithelial disk 34 includes about 50 microns, to preserve an epithelial disk 34, a separated epithelial disk 34 is rolled onto the drum 42. The drum 42 can include a diameter ranging from about 3 to about 9 mm and a length of about 12 mm. Referring also to FIG. 17, in one embodiment, to maintain integrity of the epithelial disk 34, the drum 42 can be coated with a hydrating and/or a conditioning substrate. While the layer 16 could be adhered to drum 42 without the use of a substrate, the substrate does provide controllable adhesion of layer 16 to the drum 42. The hydrating and/or conditioning substrate can include, for example, HEMA contact lenses, tissue culture media, silicone and biocompatible hydrogels. The hydrating and/or conditioning substrate can be removed from the drum after the epithelial disk 34 attaches on to the drum. Thereafter, the epithelial disk 34 can be removed from the drum 46 and replaced on the corneal surface 16, as described above.

FIG. 18 shows another embodiment of a drum 42′ that can replace the drum 42 of the device 12′ of FIGS. 14-17. The drum 42′ includes apertures 46 and a connector 48 that connects to a suction source (not shown). By applying suction to the apertures 46 of the drum 42, the epithelial disk 34 can be rolled onto the drum 42. Thereafter, the epithelial disk 34 can be removed from the drum 46 and replaced on the corneal surface 16, as described above.

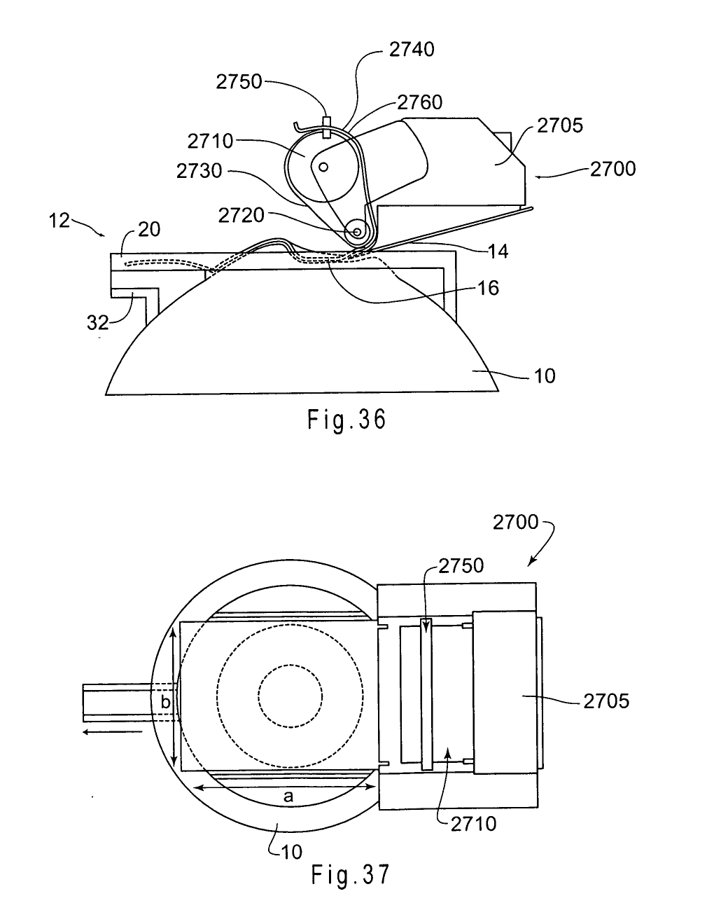

FIG. 36 shows a side view of one embodiment of a device 2700 for separating and preserving the epithelial layer 16 that has been pushed/separated by the separator devices 12 and 4000 of FIGS. 1-9 and 24-25 using a blunt edge such as shown in FIGS. 21-23 and 30-31. The device 2700 includes a body 2705, a first drum 2720 and a second drum 2730, and a belt 2730 connecting the first drum 2720 to the second drum 2730. The device 2700 accommodates a substrate, such as film 2740. Film 2740 is used to substantially preserve the epithelial layer 16 when the epithelial layer 16 is removed from the eye 10. The film 2740 can be held to the drum 2710 with a bar or clip 2750. Alternatively, the film 2740 can serve to connect the drums 2720 and 2730 and therefore eliminate the use of belt 2730.

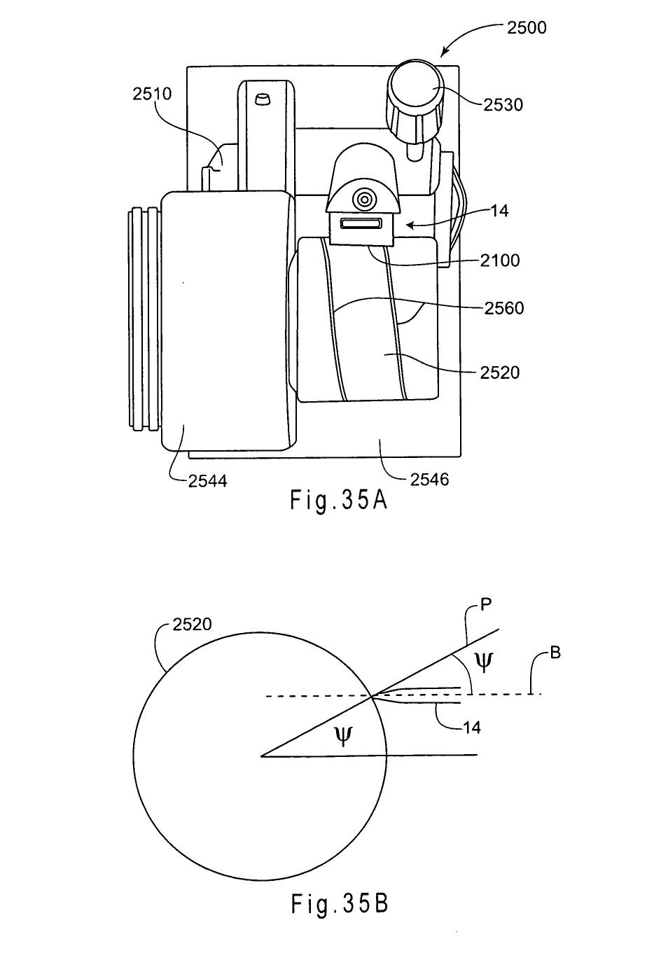

FIG. 37 shows a top view of the device 2710 and how the device 2700 is used with the clip 2750. In one embodiment, the film 2740 is rolled onto the drum 2710 and under the clip 2750 (see also FIG. 35). The first drum 2710 turns as the second drum 2720 turns since they are connected by the belt 2730. The film 2740 lays on the belt 2730 and moves as the first drum 2710 and the second drum 2720 move. The film 2740 preferably removably adheres to the belt 2730 through cohesion. The use of two drums 2710 and 2720 allows the smaller drum 2720 to go very near the separators of FIGS. 1-9, 21-23 and 30-31 and so can act as an applanator. In addition, the belt 2730 is of sufficient size to accommodate the entire removed epithelium layer 16.

The film 2740 includes an outer surface 2760. The outer surface 2760 is constructed to adhere to the epithelial layer 16 to provide mechanical stability to the epithelial layer 16 when the epithelial layer 16 is separated from the eye 10. The film 2740 includes a natural or synthetic polymer. An exemplary polymer includes HEMA (poly-2hydroxy-ethyl-methacrylate). The film 2740 includes a thickness from about 20 to about 100 micrometers. If the film 2740 is in the shape of a strip of film, a length (a) and a width (b) of the film 2740 is preferably longer and wider than the diameter of a separated epithelial layer 16.

The film 2740 is preferably hydrated to adhere the epithelial layer 16 to the film 2740. The level of hydration of the film 2740 controls adhesion to the film 2740. The hydrated film 2740 also helps to keep cracks from forming in the removed epithelial layer 16, and to help avoid the removed epithelial layer 16 from being torn or shrinking. In one embodiment, a surface of the epithelial layer 16 is dried, for example, with a sponge or with a compressed air flow. Thereafter, the film 2740 is placed on the epithelial layer 16. The epithelial layer 16 adheres to the dry film 2740 because of the difference in hydration levels between the epithelial layer and the film. Thereafter, the separator 14 is used to separate the epithelial layer 16 from the corneal substrate 18. The film 2740 and its attached epithelial layer 16 are rolled onto the first and second drums 2710, 2720.

It should be appreciated that the strip of film 2740 does not have to be applied with the device 2700 and that the strip does not need to include a coating. Moreover, the film 2740 can be applied before or after removal of the epithelial layer 16, and can be manually applied instead of using the device 2700.

The film 2740 can include other shapes such as the shape of a disc. A way to attach the epithelial layer 16 to a disc, such as a contact lens, is to separate the epithelial layer 16 from the eye 10 and remove the epithelial layer 16 to the side. The epithelial layer 16 is then smoothed with a sponge and dried with the sponge, compressed air or both. Thereafter, the removed epithelial layer 16 is placed on the film 2740. The epithelial layer 16 and the film 2740 are then dried, for example, with compressed air. After about 30 seconds of drying, the epithelial layer 16 is adhered to the film 2740 and can be more easily manipulated with a reduced risk of damage.

After the epithelial layer 16 is adhered to film 2740, the laser is applied to the surface of the cornea in a manner similar to that described previously with respect to PRK. Once the laser treatment has been completed, the corneal surface is dried and the film 2740 is laid upon the eye 10 so that the epithelial layer is laid back substantially into its original place upon the eye 10. Next, drops of water are applied to the anterior surface of the film 2740. The applied water diffuses in the film resulting in the film and the side of the film adjacent to the epithelial layer 16 being wetted. At this stage, the film 2740 is lifted off of the epithelial layer 16 and the epithelial layer 16 is attached to the eye 10.

FIG. 34 shows a perspective view of an embodiment of an exemplary machine 2500 that is used to condition a separator 14, 4018, 4018′, 4018″. The machine 2500 conditions the separator 14, 4018, 4018′, 4018″ by changing a sharp edged separator to include a generally bent edge, for example, like the front edges of the separators 14, 4018, 4018′, 4018″ shown in FIGS. 23, 25 and 30-31.

FIG. 35A shows a front view and FIG. 35B schematically shows a side view of the machine 2500 and separator 14, 4018. Referring to FIGS. 34 and 35, the machine 2500 includes a motor 2510, a rotating cylinder 2520, a weight 2530, or other way to hold the blade/separators 14, 4018 associated with leading edges 2100 and 2100′ of FIGS. 21 and 22, and a blade/separator holder 2540. The motor 2510 and a housing 2544 of the cylinder 2520 rest on a platform 2546. The separators associated with leading edges 2100 or 2100′ of FIGS. 21-22, respectively, are held by, for example, a clamp. In each case, the leading edge 2100, 2100′ is substantially parallel to the axis of rotation of cylinder 2520. As shown in FIG. 35B, the blade's plane, B, forms an angle Ψ between 0 and 20 degrees with the plane, P, defined by the axis of the cylinder 2520 and the blade's edge. The motor 2510 connects to the cylinder 2520 via a belt 2550 to rotate the cylinder 2520. In another embodiment, the motor 2510 connects directly to the cylinder 2520 to rotate the cylinder.

The cylinder 2520 includes a helical wire 2560. The helical wire 2560 and the cylinder 2520 are manufactured from steel. This helical wire 2560 serves as a helical protrusion of the rotating drum. This helical protrusion has a pitch equal to the length of the leading edge of the separator/blade. The helix causes only one point of the leading edge of the separator/blade to be conditioned at any given moment (the point of contact between the leading edge and the helical wire). As the helical wire 2560 rotates along with drum 2520, the point of contact travels along the length of the leading edge, but the amount of conditioning is equal across the entire length of the leading edge. The amount of weight 2530, and the running time and rotations of the cylinder 2520 vary the shape and width of the leading edge 2100 of the associated separator. For example, increasing the weight 2530 will result in more bending. In one embodiment, a preferred separator has been conditioned by asserting 20 mN of force on the separator to the cylinder 2520 and operating the cylinder for about 45 second at 0.7 (seven-tenths) rotations/second. The leading edge formed corresponds to leading edge 2100″ shown in FIG. 31A.

While the invention has been described above by reference to various embodiments, it will be understood that many changes and modifications can be made without departing from the scope of the invention. For example, all surfaces of the separator embodiments that contact the eye or make contact with the separated epithelial layer are smooth so that they do not cut either epithelial layer or the corneal stroma. It is therefore intended that the foregoing detailed description be understood as an illustration of the presently preferred embodiments of the invention, and not as a definition of the invention. It is only the following claims, including all equivalents, which are intended to define the scope of this invention.

We claim:

1. A separator for moving a separated portion of an eye comprising:
an upper surface having a curved shape;
a lower planar surface oriented at an angle with respect to said upper surface, wherein said upper surface and said lower planar surface define an edge therebetween having a structure to remove a portion of an eye.

2. The separator of claim 1, wherein said edge includes a structure to separate an epithelial layer of said eye from a corneal stroma of said eye without substantially damaging said epithelial layer and wherein said edge is incapable of cutting said corneal stroma.

3. The separator of claim 1, wherein said upper surface and said lower surface each does not cut said corneal stroma.

4. The separator of claim 1, wherein said edge comprises a rough texture.

5. The separator of claim 4, wherein said rough texture comprises wavy lines.

6. The separator of claim 1, wherein said curved shape substantially coincides with a portion of a circle having a radius of about 0.5 mm to 3 mm.

7. The separator of claim 1, wherein said curved shape substantially coincides with a portion of a circle having a radius of about 1.7 mm.

8. The separator of claim 7, wherein a distance from a center of said circle to a plane horizontal to said lower planar surface is less than a magnitude of said radius.

9. The separator of claim 8, wherein said distance from said center of said circle to said plane horizontal to said lower planar surface is determined so that an angle of a tangential plane to said horizontal plane has a value that ranges from about 15 degrees to about 90 degrees.

10. The separator of claim 9, wherein said angle of said tangential plane to said horizontal plane has a value of approximately 40 degrees.

11. The separator of claim 1, wherein said curved shape comprises at least one of an ellipse, a parabola, and a hyperbola.

12. A method of processing an eye of a patient for a corrective procedure comprising:
providing a separator having an edge;
penetrating an epithelial layer of said eye of said patient with said edge;
moving said separator relative said eye of said patient; and
separating at least a portion of said epithelial layer as said separator moves, wherein said at least a portion of said separated epithelial layer is rolled during said separating.

13. The method of claim 12, wherein said separated epithelial layer remains in place when said separator is retracted away from said separated epithelial layer.

15. The method of claim 12, wherein there is no applanation performed separate from said leading edge.

15. The method of claim 12, wherein said separated epithelial layer remains attached to said eye by at least one end.

16. The method of claim 12, wherein the separator begins moving at a first position and travels away from said first position before being retracted.

17. The method of claim 16, wherein said separated epithelial layer remains at a second position away from said first position without needing to be manipulated as said separator is retracted.

18. The method of claim 17, wherein said second position is away from said first position by at least half the distance traveled by said separator in one direction.

19. A separator for moving a separated portion of an eye comprising:
an upper surface having two planar surfaces that meet each other;
a lower planar surface oriented at an angle with respect to said upper surface, wherein said upper surface and said lower planar surface define an edge therebetween having a structure to remove a portion of an eye.

20. The separator of claim 19, wherein said two planar surfaces form a side-wise V-shape.

21. The separator of claim 19, wherein an angle between said two planar surfaces comprises about 0 degrees to about 180 degrees.

22. The separator of claim 19, wherein a distance from a separating edge and a point of intersection of said two planar surfaces ranges from about 15 microns to about 4 mm.

23. The separator of claim 19, wherein said upper surface comprises a flat portion having a height h of about 0.5 to about 2 mm.

24. The separator of claim 23, wherein said upper surface may be positioned at an angle from about 40 degrees to about 90 degrees from a surface parallel to an axis of travel of said separator along said eye.

25. The separator of claim 19, wherein said upper surface comprises a generally convex shape with regard to a direction of moving said separated portion of said eye.

26. The separator of claim 19, wherein said upper surface comprises a lip to push down said separated portion of said eye.

27. The separator of claim 19, wherein said upper surface comprises a structure adapted to roll said separated portion of said eye.

28. A separator for moving a separated portion of an eye comprising:
an upper surface; and
a lower planar surface oriented at an angle with respect to said upper surface, wherein said upper surface and said lower planar surface define an edge therebetween, wherein said upper surface includes a structure to receive said removed portion of said eye and to roll said removed portion.
